SEQUENCE LISTING (14 sequences)

SEQ ID NO: 1 - protein, 12 aa, synthetic construct
  REGION 1..12: note = Peptide having affinity to IgG
EPIHRSTLTALL

SEQ ID NO: 2 - protein, 10 aa, synthetic construct
  REGION 1..10: note = Peptide having affinity to IgG
FARLVSSIRY

SEQ ID NO: 3 - protein, 10 aa, synthetic construct
  REGION 1..10: note = Peptide having affinity to IgG
FGRLVSSIRY

SEQ ID NO: 4 - protein, 10 aa, synthetic construct
  REGION 1..10: note = Peptide having affinity to IgG
TWKTSRISIF

SEQ ID NO: 5 - protein, 4 aa, synthetic construct
  REGION 1..4: note = Peptide having affinity to IgG
QSYP

SEQ ID NO: 6 - protein, 6 aa, synthetic construct
  REGION 1..6: note = Peptide having affinity to IgG
HWRGWV

SEQ ID NO: 7 - protein, 6 aa, synthetic construct
  REGION 1..6: note = Peptide having affinity to IgG
HYFKFD

SEQ ID NO: 8 - protein, 6 aa, synthetic construct
  REGION 1..6: note = Peptide having affinity to IgG
HFRRHL

SEQ ID NO: 9 - protein, 4 aa, synthetic construct
  REGION 1..4: note = Peptide having affinity to IgG
DAAG

SEQ ID NO: 10 - protein, 8 aa, synthetic construct
  REGION 1..8: note = Peptide having affinity to IgG
NARKFYKG

SEQ ID NO: 11 - protein, 8 aa, synthetic construct
  REGION 1..8: note = Peptide having affinity to IgG
NKFRGKYK

SEQ ID NO: 12 - protein, 17 aa, synthetic construct
  REGION 1..17: note = Peptide having affinity to IgG
RGNCAYHKGQIIWCTYH

SEQ ID NO: 13 - protein, 17 aa, synthetic construct
  REGION 1..17: note = Peptide having affinity to IgG
RGNCAYHKGQLVWCTYH

SEQ ID NO: 14 - protein, 34 aa, synthetic construct
  REGION 1..34: note = Peptide having affinity to IgG
FNKQCQRRFYEALHDPNLNEEQRNARIRSIRDDC

(19) United States
(12) Patent Application Publication
(10) Pub. No.: US 2024/0218015 A1
(43) Pub. Date: Jul. 4, 2024

(54) METHODS FOR PRODUCING INTERMEDIATE ANTIBODIES REGIOSELECTIVELY MODIFIED, AND ANTIBODY DERIVATIVES REGIOSELECTIVELY HAVING BIOORTHOGONAL FUNCTIONAL GROUP OR FUNCTIONAL SUBSTANCE

(71) Applicant: Ajinomoto Co., Inc., Tokyo (JP)

(72) Inventors: Yutaka MATSUDA, Kawasaki-shi (JP); Yuichi NAKAHARA, Kawasaki-shi (JP); Kei YAMADA, Kawasaki-shi (JP); Keisuke KATO, Kawasaki-shi (JP); Yuta ENDO, Kawasaki-shi (JP); Brian Alan MENDELSOHN, San Diego, CA (US)

(73) Assignee: Ajinomoto Co., Inc., Tokyo (JP)

(21) Appl. No.: 18/598,901

(22) Filed: Mar. 7, 2024

Related U.S. Application Data
(63) Continuation of application No. PCT/JP2022/033613, filed on Sep. 7, 2022.
(60) Provisional application No. 63/241,577, filed on Sep. 8, 2021.

(51) Int. Cl. C07K 1/04; C07K 1/02

(52) U.S. Cl. CPC C07K 1/045; C07K 1/02

(57) ABSTRACT

A method for producing an intermediate antibody regioselectively modified, which comprises:
(1) mixing a solution comprising an antibody material and a solution comprising a reagent for regioselectively modifying an antibody in a micromixer to form a mixture comprising the antibody material and the reagent,
wherein the reagent comprises a compound comprising an affinity substance to an antibody and a reactive group to an antibody; and
(2) passing the mixture through a reaction channel to allow the antibody material and the reagent to react in the reaction channel to form a solution comprising an intermediate antibody regioselectively modified,
wherein the processes (1) and (2) are continuously performed in a flow microreactor,
is capable of rapidly producing a desired antibody derivative regioselectively having a functional substance or functional substances.

### CROSS REFERENCES TO RELATED APPLICATIONS

This application is a continuation of International Patent Application No. PCT/JP2022/033613, filed on Sep. 7, 2022, and claims priority to U.S. Provisional Patent Application No. 63/241,557, filed on Sep. 8, 2021, 2003, both of which are incorporated herein by reference in their entireties.

### REFERENCE TO A SEQUENCE LISTING

In accordance with 37 CFR § 1.833-1835 and 37 CFR § 1.77(b) (5), the specification makes reference to a Sequence Listing submitted electronically as a .xml file named “552222US_ST26.xml”. The .xml file was generated on Feb. 6, 2024, and is 18,689 bytes in size. The entire contents of the Sequence Listing are hereby incorporated by reference.

### BACKGROUND OF THE INVENTION

### Field of the Invention

The present invention relates to methods for producing intermediate antibodies regioselectively modified, and antibody derivatives regioselectively having bioorthogonal functional group(s) or functional substance(s).

### Discussion of the Background

In a reaction of a plurality of molecules in a liquid phase system, different solutions including different molecules are mixed before the reaction. The mixing time required for obtaining a homogeneous solution depends on the diffusion distance of molecules in the solution. The mixing in a batch process (for example, mixing with a stirrer bar in a flask), the diffusion distance of molecules is large and it takes at least a few seconds to obtain a homogeneous solution by mixing of different solutions. Thus, in the batch process, the mixing takes time and it is difficult to allow a plurality of molecules to react in a short time on the order of less than a second. In order to allow a plurality of molecules to react in a short time, quick mixing of a plurality of components is necessary.

A flow microreactor (FMR), which may be simply referred to as microreactor, is a flow reactor in which a reaction proceeds in microchannels. FMRs are mainly used in organic synthesis of low-molecular organic compounds. In the FMR, a micromixer having a microstructure is used to enable mixing and reaction of a plurality of components in a minute environment.

Mixing of solutions by the micromixer in the FMR is mixing in minute channels, and the diffusion distance of molecules is small. The FMR therefore rapidly yields a homogeneous solution by speeding up mixing with the micromixer and allows a plurality of components to react in a short time on the order of less than a second.

In the micromixer in the FMR, a variety of mixing types can be used. Examples of mixing types of micromixers include a sheath flow type in which a plurality of solutions flowing in the forward direction merge, a static type in which mixing is promoted by a structure in a channel after a plurality of solutions merge, a helix type in which mixing is performed by forming a three-dimensional spiral, and a multilayer flow type in which a plurality of channels are merged such that a plurality of solutions flow alternately at short intervals. The micromixer has a feature corresponding to its mixing type. For example, in the sheath flow type, first and second solutions flowing in the forward direction are merged by inserting a tube of the second solution into a tube in which the first solution flows. The sheath flow mixer having such a feature is suitable for mixing a plurality of solutions with a significant difference in flow rate, but its mixing speed is not high. The static type tends to avoid failures such as clogging of a channel, but the degree of mixing at the time of contact between a plurality of solutions is not high.

FMRs have recently been used in synthesis of not only low-molecular organic compounds but also antibody-drug conjugates (ADCs).

For example, WO2020/075817, which is incorporated herein by reference in its entirety, describes a method of synthesizing an ADC including the following processes, characterized by using an inhibitor for a reducing agent for controlling the value of a drug-to-antibody ratio (DAR) of the ADC (see Examples):
(1) mixing an IgG antibody and a reducing agent to partially reduce the IgG antibody (antibody derivatization reaction); and
(2) mixing (A) a solution containing (a1) the reducing agent, (a2) the partially reduced IgG antibody, and (a3) a linker linked with a drug with (B) a solution containing an inhibitor for the reducing agent to link the antibody to the drug through the linker (conjugation reaction).

US Patent Application Publication No. 2019/0270769, which is incorporated herein by reference in its entirety, describes a method of preparing an ADC composition, the method including continuously performing the following processes, characterized by using single-pass tangential flow filtration (SPTFF) for concentration of the ADC and removal of unconjugated products (specifically, unconjugated drug):
(1) derivatizing a drug to enable a reaction with an amino group in a lysine residue side chain in an antibody (drug derivatization reaction);
(2) allowing the drug derivatized to react with an amino group in a lysine residue side chain in the antibody to react with the antibody to generate an ADC (in which the antibody and the drug are conjugated through the amino group in the lysine residue side chain in the antibody) (conjugation reaction); and
(3) purifying the ADC through single-pass tangential flow filtration (SPTFF).

WO2019/016067 and Meaghan M. Sebeika et al., J. Flow Chem. 2015, 5(3), 151-154, which are incorporated herein by reference in their entireties, describe a method of synthesizing an ADC, characterized by performing the following process using a microreactor (see Examples):
(1) allowing a drug derivatized in advance to react with an amino group in a lysine residue side chain in an antibody to react with the antibody to generate an ADC (in which the antibody and the drug are conjugated through the amino group in the lysine residue side chain in the antibody) (drug derivatization reaction).

### SUMMARY OF THE INVENTION

Accordingly, it is an object of the present invention to provide a technology capable of rapidly producing a desired antibody derivative regioselectively having a functional substance or functional substances.

This and other objects, which will become apparent during the following detailed description, have been achieved by the inventors' discovery that a desired intermediate antibody and antibody derivative can be produced regioselectively and rapidly using a compound comprising an affinity substance to an antibody and a reactive group to an antibody in an FMR.

As far as the inventors of the present invention know, there have been no report on regioselective modification reactions by FMRs (in particular, regioselective modification reactions in biological polymers such as antibodies) or the use of affinity substances in FMRs (in particular, the use of affinity substances to biological polymers such as antibodies). A reaction that requires association by an affinity substance to a target has been thought to be a thermodynamics dominant reaction and unsuitable for FMRs that are kinetics dominant. In particular, when a high level of control over reactions is desired as in substances that require high-level regioselective modification and modification ratios, the reaction requiring association by an affinity substance is thought to be unsuitable for FMRs. However, the inventors of the present invention have found that a regioselective intermediate antibody and antibody derivative can be produced by using the above compound in an FMR and that various advantages including rapid synthesis can be achieved in the production, and has led to completion of the invention.

Accordingly, the present invention is as follows.
(1) A method for producing an intermediate antibody regioselectively modified, the method comprising:
   (1) mixing a solution comprising an antibody material and a solution comprising a reagent for regioselectively modifying an antibody in a micromixer to form a mixture comprising the antibody material and the reagent,
   wherein the reagent comprises a compound comprising an affinity substance to an antibody and a reactive group to an antibody; and
   (2) passing the mixture through a reaction channel to allow the antibody material and the reagent to react in the reaction channel to form a solution comprising an intermediate antibody regioselectively modified,
   wherein the processes (1) and (2) are continuously performed in a flow microreactor.
(2) The method according to (1), wherein the antibody material is IgG.
(3) The method according to (1) or (2), wherein the affinity substance is a peptide having affinity to a heavy chain constant region of an antibody.
(4) The method according to any of (1) to (3), wherein the reactive group is a reactive group to an amino group.
(5) The method according to any of (1) to (4), wherein the compound comprises the affinity substance, the reactive group, and a cleavable site, and optionally further comprises a bioorthogonal functional group.
(6) The method according to (5), wherein the compound comprises the affinity substance, the reactive group, the cleavable site, and the bioorthogonal functional group, wherein
   (i) the cleavable site is present at an affinity substance-side position between the affinity substance and the reactive group, and
   (ii) the bioorthogonal functional group is present at a reactive group-side position between the affinity substance and the reactive group.
(7) The method according to (5), wherein the compound comprises the affinity substance, the reactive group, and a cleavable site capable of forming a bioorthogonal functional group by cleavage,
   wherein the cleavable site capable of forming a bioorthogonal functional group by cleavage is present at a position between the affinity substance and the reactive group.
(8) The method according to any of (1) to (4), wherein the compound comprises the affinity substance, the reactive group, a leaving group, and a bioorthogonal functional group, wherein
   (i) the leaving group and the reactive group are coupled with each other and present at a position between the affinity substance and the bioorthogonal functional group,
   (ii) the leaving group is present at an affinity substance-side position between the affinity substance and the bioorthogonal functional group, and
   (iii) the reactive group is present at a bioorthogonal functional group-side position between the affinity substance and the bioorthogonal functional group.
(9) The method according to any of (1) to (8), wherein the micromixer is a collision-type micromixer.
(10) The method according to (9), wherein the collision-type micromixer is a T-shaped micromixer.
(11) The method according to any of (1) to (10), wherein
   the solution comprising an antibody material is passed through a first inlet channel,
   the solution comprising a reagent for regioselectively modifying an antibody is passed through a second inlet channel,
   the micromixer is provided at a junction of the first inlet channel and the second inlet channel, and
   both of a representative diameter ratio of the micromixer to the first inlet channel (micromixer/first inlet channel) and a representative diameter ratio of the micromixer to the second inlet channel (micromixer/second inlet channel) are 0.95 or less.
(12) The method according to any of (1) to (11), wherein a residence time of the mixture in the reaction channel is less than 3 minutes.
(13) The method according to any of (1) to (12), wherein a modification ratio of the compound to the intermediate antibody (modification by the compound/antibody) is 1.5 to 2.5 per immunoglobulin unit comprising two light chains and two heavy chains.
(14) The method according to any of (1) to (13), wherein a monomer ratio of the intermediate antibody analyzed by size exclusion chromatography is 98% or more.
(15) A method for producing an antibody derivative regioselectively having a bioorthogonal functional group or bioorthogonal functional groups, the method comprising:
   (1) mixing a solution comprising an antibody material and a solution comprising a reagent for regioselectively modifying an antibody in a first micromixer to form a first mixture comprising the antibody material and the reagent,
   wherein the reagent comprises a compound comprising an affinity substance to an antibody, a reactive group to an antibody, and a cleavable site, and optionally further comprising a bioorthogonal functional group;
   (2) passing the first mixture through a first reaction channel to allow the antibody material and the reagent to react in the first reaction channel to form a solution comprising an intermediate antibody regioselectively modified,
   wherein the intermediate antibody regioselectively modified is an intermediate antibody regioselectively having a structural unit comprising the affinity substance and the cleavable site and optionally further comprising a bioorthogonal functional group;
   (3) mixing the solution comprising the intermediate antibody and a solution comprising a cleavage agent in a second micromixer to form a second mixture comprising the intermediate antibody and the cleavage agent; and
   (4) passing the second mixture through a second reaction channel to allow the intermediate antibody and the cleavage agent to react in the second reaction channel to form a solution comprising an antibody derivative regioselectively having a bioorthogonal functional group or bioorthogonal functional groups,
   wherein the bioorthogonal functional group in the antibody derivative is (a) the bioorthogonal functional group in the compound or (b) a bioorthogonal functional group formed by cleavage of the cleavable site by the cleavage agent,
   wherein the processes (1) to (4) are continuously performed in a flow microreactor.
(16) The method according to (15), wherein a total time of a residence time of the first mixture in the first reaction channel and a residence time of the second mixture in the second reaction channel is within 4.5 minutes.
(17) The method according to (15) or (16), wherein a modification ratio of the bioorthogonal functional group to the antibody derivative regioselectively having a bioorthogonal functional group or bioorthogonal functional groups (bioorthogonal functional group/antibody) is 1.5 to 2.5 per immunoglobulin unit comprising two light chains and two heavy chains.
(18) The method according to any of (15) to (17), wherein a monomer ratio of the antibody derivative regioselectively having a bioorthogonal functional group or bioorthogonal functional groups analyzed by size exclusion chromatography is 98% or more.
(19) A method for producing an antibody derivative regioselectively having a functional substance or functional substances, the method comprising:
   (I) forming (a) a solution comprising an intermediate antibody regioselectively having a bioorthogonal functional group by the method according to (8) or forming (b) a solution comprising an antibody derivative regioselectively having a bioorthogonal functional group or bioorthogonal functional groups by the method according to (15);
   (II) mixing the solution formed in (I) and a solution comprising a functional substance in a micromixer to form (a) a mixture comprising the intermediate antibody and the functional substance or (b) a mixture comprising the antibody derivative and the functional substance; and
   (III) passing the mixture formed in (II) through a reaction channel to allow (a) the intermediate antibody and the functional substance or (b) the antibody derivative and the functional substance to react in the reaction channel to form a solution comprising an antibody derivative regioselectively having a functional substance or functional substances,
   wherein the processes (I) to (III) are continuously performed in a flow microreactor.
(20) The method according to (19), wherein the functional substance is a pharmaceutical, a labelling substance, or a stabilizer.
(21) The method according to (19) or (20), wherein a total residence time in all reaction channels is within 6 minutes.
(22) The method according to any of (19) to (21), wherein a modification ratio of the functional substance to the antibody derivative regioselectively having a functional substance or functional substances (functional substance/antibody) is 1.5 to 2.5 per immunoglobulin unit comprising two light chains and two heavy chains.
(23) The method according to any of (19) to (22), wherein a monomer ratio of the antibody derivative regioselectively having a functional substance or functional substances analyzed by size exclusion chromatography is 98% or more.

### Effect of the Invention

According to the methods of the present invention, a desired intermediate antibody and antibody derivative can be produced regioselectively and rapidly.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 is a diagram illustrating an overview of regioselective modification of an antibody by a compound. First, the compound is associated with the antibody through an affinity substance to an antibody. Next, the compound reacts with a desired group in the antibody (a functional group in a side chain of a specific amino acid residue in a target region of the antibody present in the vicinity of an association site between the affinity substance and the antibody. For example, in FIG. 1, an amino group in a side chain of a lysine residue. The same applies in the following drawings.) through a reactive group (in FIG. 1, activated ester. The same applies in the following drawings) to form an intermediate antibody regioselectively modified. There are cases in which the affinity substance is introduced to the antibody (e.g., see FIGS. 2-1 and 3-1) and in which the affinity substance is not introduced to the antibody (e.g., see FIG. 4), depending on the kind of compounds. Representative examples of a linker and a modification group are as illustrated in FIGS. 2-1, 3-1, and 4.

FIG. 2-1 is a diagram illustrating an example of a process (2) in production of an intermediate antibody regioselectively modified, using a compound comprising an affinity substance to an antibody, a reactive group to an antibody, a cleavable site, and a bioorthogonal functional group (e.g., see WO 2018/199337, WO 2019/240287, and WO 2020/090979, which are incorporated herein by reference in their entireties). First, the compound is associated with the antibody through an affinity substance to the antibody, and then the reactive group in the compound reacts with a desired group in the antibody. By this reaction, a specific structural unit can be regioselectively added to the antibody, so that an intermediate antibody regioselectively having a specific structural unit can be formed.

FIG. 2-2 is a diagram illustrating an example of a process (4) in production of an antibody derivative regioselectively having a bioorthogonal functional group or bioorthogonal functional groups, using a compound comprising an affinity substance to an antibody, a reactive group to an antibody, a cleavable site, and a bioorthogonal functional group. By cleavage of the cleavable site, an antibody derivative regioselectively having a bioorthogonal functional group or bioorthogonal functional groups is formed from an intermediate antibody regioselectively having a specific structural unit. When the specific structural unit comprises a cleavable site and a bioorthogonal functional group in addition to an affinity substance to an antibody, the affinity substance can be eliminated from the antibody by cleavage of the cleavable site, so that an antibody derivative regioselectively having a bioorthogonal functional group or bioorthogonal functional groups (an antibody derivative that does not comprise an affinity substance or a cleavable site) can be formed.

FIG. 3-1 is a diagram illustrating another example of the process (2) in production of an intermediate antibody regioselectively modified, using a compound comprising an affinity substance to an antibody, a reactive group to an antibody, and a cleavable site capable of forming a bioorthogonal functional group by cleavage (e.g., see WO 2018/199337, WO 2019/240287, and WO 2020/090979, which are incorporated herein by reference in their entireties). First, the compound is associated with an antibody through the affinity substance to the antibody, and then the reactive group in the compound reacts with a desired group in the antibody. By this reaction, a specific structural unit can be regioselectively added to the antibody, so that an intermediate antibody regioselectively having a specific structural unit can be formed.

FIG. 3-2 is a diagram illustrating another example of the process (4) in production of an antibody derivative regioselectively having a bioorthogonal functional group or bioorthogonal functional groups, using a compound comprising an affinity substance to an antibody, a reactive group to an antibody, and a cleavable site capable of forming a bioorthogonal functional group by cleavage. By cleavage of such a cleavable site, an antibody derivative regioselectively having a bioorthogonal functional group or bioorthogonal functional groups is formed from an intermediate antibody regioselectively having a specific structural unit. Even when the specific structural unit does not comprise a bioorthogonal functional group, a cleavable site capable of forming a bioorthogonal functional group by cleavage is used as a cleavable site, whereby the affinity substance can be eliminated from the antibody while the cleavable site is converted into a bioorthogonal functional group by cleavage of the cleavable site, so that an antibody derivative regioselectively having a bioorthogonal functional group or bioorthogonal functional groups (an antibody derivative that does not comprise an affinity substance or a cleavable site) can be formed.

FIG. 4 is a diagram illustrating still another example of the process (2) in production of an intermediate antibody regioselectively modified, using a compound comprising an affinity substance to an antibody, a reactive group (electrophilic group) and a leaving group coupled therewith, and a bioorthogonal functional group (e.g., see WO 2019/240288, which is incorporated herein by reference in its entirety). First, the compound is associated with an antibody through the affinity substance to the antibody, and then the reactive group (electrophilic group) in the compound reacts with a desired nucleophilic group in the antibody (a functional group in a side chain of a specific amino acid residue in a target region of the antibody present in the vicinity of an association site between the affinity substance and the antibody). By this reaction, the bioorthogonal functional group can be regioselectively added to the antibody while a partial structure comprising the affinity substance and a group comprising the leaving group is eliminated from the compound, so that an intermediate antibody regioselectively having a bioorthogonal functional group or bioorthogonal functional groups can be formed in a single step.

FIG. 5 is a diagram illustrating an overview of production of an antibody derivative regioselectively having a functional substance or functional substances. An antibody regioselectively having a bioorthogonal functional group or bioorthogonal functional groups can react with a functional substance through the bioorthogonal functional group. By this reaction, an antibody derivative regioselectively having a functional substance or functional substances (e.g., ADC) can be formed.

FIG. 6 is a diagram illustrating an exemplary configuration of an FMR that can be used in the method of the present invention.

FIG. 7 is a diagram illustrating another exemplary configuration of an FMR that can be used in the method of the present invention.

FIG. 8 is a diagram illustrating still another exemplary configuration of an FMR that can be used in the method of the present invention.

### DETAILED DESCRIPTION OF THE PREFERRED EMBODIMENTS

### 1. Method for Producing Intermediate Antibody Regioselectively Modified

### 1-1. Overview

The present invention provides a method for producing an intermediate antibody regioselectively modified. The method according to the present invention includes the following (1) and (2):
(1) mixing a solution comprising an antibody material and a solution comprising a reagent for regioselectively modifying an antibody in a micromixer to form a mixture comprising the antibody material and the reagent,
wherein the reagent comprises a compound comprising an affinity substance to an antibody and a reactive group to an antibody; and
(2) passing the mixture through a reaction channel to allow the antibody material and the reagent to react in the reaction channel to form a solution comprising an intermediate antibody regioselectively modified.

The processes (1) and (2) are continuously performed in a flow microreactor (FMR).

The detail of the processes (1) and (2) will be described in order below.

### 1-2. Process (1)

The process (1) can be performed, for example, by introducing a solution comprising an antibody material into a first inlet channel and introducing a solution comprising a reagent for regioselectively modifying an antibody into a second inlet channel so that both solutions are mixed in a micromixer at a junction of the first inlet channel and the second inlet channel. By such mixing, a mixture comprising the antibody material and the reagent is formed. The solutions can be introduced into the first and second inlet channels, for example, by feeding the solution from a reservoir or by passing the solution from an upstream channel (for example, passing the solution from a junction channel of a first upstream channel and a second upstream channel). For example, the solution feeding can be performed using a pump. The solution passing from an upstream channel can be promoted by feeding the solution from an upstream reservoir to the upstream channel using a pump.

The antibody material used in the process (1) is not specifically limited as long as it is any antibody of which derivatization is desired and may be either an unmodified antibody or a modified antibody. When the antibody material is an unmodified antibody, a solution comprising an unmodified antibody can be introduced into the first inlet channel from a reservoir. When the antibody material is a modified antibody, a solution comprising a modified antibody may be introduced into the first inlet channel from a reservoir, or a solution comprising a modified antibody may be introduced from an outlet channel of a modified-antibody-forming system present upstream of the first inlet channel (for example, a channel system comprising a first upstream inlet channel for introducing an unmodified antibody, a second upstream inlet channel comprising a modification reagent for the unmodified antibody, a micromixer at the junction of these channels, and an upstream reaction channel for allowing the unmodified antibody and the modification reagent to react to form a modified antibody).

The term “antibody” in antibody-related expressions such as an antibody material, and an intermediate antibody and an antibody derivative formed from an antibody material is defined as follows.

For example, the antibody may be derived from, but not limited to, animals such as mammals and birds (for example, chickens). Preferably, an immunoglobulin unit is derived from mammals. Examples of the mammals include primates (e.g., humans, monkeys, and chimpanzees), rodents (e.g., mice, rats, guinea pigs, hamsters, and rabbits), pets (e.g., dogs and cats), domestic animals (e.g., cattle, pigs, and goats), and working animals (e.g., horses and sheep). The mammals are preferably primates or rodents, more preferably humans.

The kind of antibody may be a polyclonal antibody or a monoclonal antibody. The antibody may be a divalent antibody (e.g., IgG, IgD, and IgE) or may be a tetra- or more valent antibody (e.g., IgA antibody and IgM antibody). Preferably, the antibody is a monoclonal antibody. Examples of the monoclonal antibody include chimeric antibodies, humanized antibodies, human antibodies, antibodies with a predetermined glycan attached (e.g., antibodies modified to have a glycan-binding consensus sequence such as an N-glycan-binding consensus sequence), bispecific antibodies, Fc region proteins, and Fc fusion proteins. Examples of the isotype of the monoclonal antibody include IgG (e.g., IgG1, IgG2, IgG3, and IgG4), IgM, IgA, IgD, IgE, and IgY. In the present invention, a full-length antibody or an antibody fragment including a variable region and a CH1 domain and a CH2 domain can be used as the monoclonal antibody, but a full-length antibody is preferred. The antibody is preferably an IgG monoclonal antibody, and more preferably an IgG full-length monoclonal antibody.

Any antigen can be used as an antigen for the antibody. Examples of such an antigen include proteins [including oligopeptides and polypeptides and which may be a protein modified by a biomolecule such as sugar (e.g., glycoprotein)], glycans, nucleic acids, and low-molecular compounds. Preferably, the antibody may be an antibody for a protein as an antigen. Examples of the protein include membrane receptors, membrane proteins other than membrane receptors (e.g., extracellular matrix proteins, channel proteins, and transporter proteins), ligands, and soluble receptors.

More specifically, the protein serving as an antigen for the antibody may be a disease target protein. Examples of the disease target protein include those below.

### (1) Cancerous Regions

PD-Li, GD2, PDGFRα (platelet-derived growth factor receptor), CD22, HER2, phosphatidylserine (PS), EpCAM, fibronectin, PD-1, VEGFR-2, CD33, HGF, gpNMB, CD27, DEC-205, folate receptor, CD37, CD19, Trop2, CEACAM5, SiP, HER3, IGF-1R, DLL4, TNT-1/B, CPAAs, PSMA, CD20, CD105 (endoglin), ICAM-1, CD30, CD16A, CD38, MUC1, EGFR, KIR2DL1, 2, NKG2A, tenascin-C, IGF (insulin-like growth factor), CTLA-4, mesothelin, CD138, c-Met, Ang2, VEGF-A, CD79b, ENPD3, folate receptor α, TEM-1, GM2, glypican 3, macrophage inhibitory factor, CD74, Notch1, Notch2, Notch3, CD37, TLR-2, CD3, CSF-1R, FGFR2b, HLA-DR, GM-CSF, EphA3, B7-H3, CD123, gpA33, Frizzled7 receptor, DLL4, VEGF, RSPO, LIV-1, SLITRK6, Nectin-4, CD70, CD40, CD19, SEMA4D (CD100), CD25, MET, Tissue Factor, IL-8, EGFR, cMet, KIR3DL2, Bst1 (CD157), P-cadherin, CEA, GITR, TAM (tumor associated macrophage), CEA, DLL4, Ang2, CD73, FGFR2, CXCR4, LAG-3, GITR, Fucosyl GM1, IGF-1, Angiopoietin 2, CSF-1R, FGFR3, OX40, BCMA, ErbB3, CD137 (4-1BB), PTK7, EFNA4, FAP, DR5, CEA, Ly6E, CA6, CEACAM5, LAMP1, tissue factor, EPHA2, DR5, B7-H3, FGFR4, FGFR2, α2-PI, A33, GDF15, CAIX, CD166, ROR1, GITR, BCMA, TBA, LAG-3, EphA2, TIM-3, CD-200, EGFRvIII, CD16A, CD32B, PIGF, Axl, MICA/B, Thomsen-Friedenreich, CD39, CD37, CD73, CLEC12A, Lgr3, transferrin receptor, TGFβ, IL-17, 5T4, RTK, Immune Suppressor Protein, NaPi2b, Lewis blood group B antigen, A34, Lysil-Oxidase, DLK-1, TROP-2, α9 integrin, TAG-72(CA72-4), CD70

### (2) Autoimmune Diseases/Inflammatory Diseases

IL-17, IL-6R, IL-17R, INF-α, IL-5R, IL-13, IL-23, IL-6, ActRIIB, β7-Integrin, IL-4αR, HAS, Eotaxin-1, CD3, CD19, TNF-α, IL-15, CD3c, Fibronectin, IL-1B, IL-la, IL-17, TSLP (Thymic Stromal Lymphopoietin), LAMP (Alpha4 Beta 7 Integrin), IL-23, GM-CSFR, TSLP, CD28, CD40, TLR-3, BAFF-R, MAdCAM, IL-31R, IL-33, CD74, CD32B, CD79B, IgE (immunoglobulin E), IL-17A, IL-17F, C5, FcRn, CD28, TLR4, MCAM, B7RP1, CXCR1, 2 Ligands, IL-21, Cadherin-11, CX3CL1, CCL20, IL-36R, IL-10R, CD86, TNF-α, IL-7R, Kv1.3, α9 integrin, LIFHT

### (3) Central Nerve Diseases

CGRP, CD20, β amyloid, β amyloid protofibrin, Calcitonin Gene-Related Peptide Receptor, LINGO (Ig Domain Containing 1), α-synuclein, extracellular tau, CD52, insulin receptor, tau protein, TDP-43, SOD1, TauC3, JC virus

### (4) Infectious Diseases

Clostridium Difficile toxin B, cytomegalovirus, RS virus, LPS, S. Aureus Alpha-toxin, M2e protein, Psl, PcrV, S. aureus toxin, influenza A, Alginate, Staphylococcus aureus, PD-Li, influenza B, Acinetobacter, F-protein, Env, CD3, pathogenic Escherichia coli, Klebsiella, Streptococcus pneumoniae

### (5) Genetic and Rare Diseases

amyloid AL, SEMA4D (CD100), insulin receptor, ANGPTL3, IL4, IL13, FGF23, adrenocorticotropic hormone, transthyretin, huntingtin

### (6) Eye Diseases

Factor D, IGF-1R, PGDFR, Ang2, VEGF-A, CD-105 (Endoglin), IGF-1R, β amyloid

### (7) Fields of Bone and Orthopedics

Sclerostin, Myostatin, Dickkopf-1, GDF8, RNAKL, HAS, Siglec-15

### (8) Blood Diseases

vWF, Factor IXa, Factor X, IFNγ, C5, BMP-6, Ferroportin, TFPI

### (9) Other Diseases

BAFF (B cell activating factor), IL-1β, PCSK9, NGF, CD45, TLR-2, GLP-1, TNFR1, C5, CD40, LPA, prolactin receptor, VEGFR-1, CB1, Endoglin, PTH1R, CXCL1, CXCL8, IL-1B, AT2-R, IAPP

Specific examples of the monoclonal antibody include specific chimeric antibodies (e.g., rituximab, basiliximab, infliximab, cetuximab, siltuximab, dinutuximab, and altertoxaximab), specific humanized antibodies (e.g., daclizumab, palivizumab, trastuzumab, alemtuzumab, omalizumab, efalizumab, bevacizumab, natalizumab (IgG4), tocilizumab, eculizumab (IgG2), mogamulizumab, pertuzumab, obinutuzumab, vedolizumab, pembrolizumab (IgG4), mepolizumab, elotuzumab, daratumumab, ixekizumab (IgG4), reslizumab (IgG4), atezolizumab), and specific human antibodies (e.g., adalimumab (IgG1), panitumumab, golimumab, ustekinumab, canakinumab, ofatumumab, denosumab (IgG2), ipilimumab, belimumab, raxibacumab, ramucirumab, nivolumab, dupilumab (IgG4), secukinumab, evolocumab (IgG2), alirocumab, necitumumab, brodalumab (IgG2), and olaratumab) (those that do not refer to IgG subtypes are IgG1).

For the positions of amino acid residues in an antibody and the position of the constant region of the heavy chain (e.g., CH2 domain), see EU numbering (http://www.imgt.org/IMGTScientificChart/Numbering/Hu_IGHGnber.html). For example, when human IgG is a target, the lysine residue in position 246 corresponds to the 16th amino acid residue in the human IgG CH2 region, the lysine residue in position 248 corresponds to the 18th amino acid residue in the human IgG CH2 region, the lysine residue in position 288 corresponds to the 58th amino acid residue in the human IgG CH2 region, the lysine residue in position 290 corresponds to the 60th amino acid residue in the human IgG CH2 region, and the lysine residue in position 317 corresponds to the 87th amino acid residue in the human IgG CH2 region. The notation “position 246/248” indicates that the lysine residue in position 246 or position 248 is the target. The notation “position 288/290” indicates that the lysine residue in position 288 or position 290 is the target.

In the present invention, “regioselectively” or “regioselectivity” means a state in which even though a specific amino acid residue is not present locally at a specific region in the antibody, a certain structural unit capable of binding to a specific amino acid residue in the antibody is present locally at a specific region in the antibody. The expressions related to regioselectivity, such as “regioselectively having”, “regioselective binding”, “binding with regioselectivity” mean that the possession rate or the binding rate of a certain structural unit in the target region comprising one or more specific amino acid residues is higher at a significant level than the possession rate or the binding rate of the structural unit in the non-target region comprising a plurality of amino acid residues homogeneous with respect to the specific amino acid residues in the target region. Such regioselectivity may be 50% or more, preferably 60% or more, more preferably 70% or more, even more preferably 80% or more, and particularly preferably 90% or more, 95% or more, 96% or more, 97% or more, 98% or more, 99% or more, 99.5% or more, or 100%.

In the present invention, a specific amino acid residue in an intermediate antibody or an antibody derivative (e.g., a specific amino acid residue in a heavy chain) can be regioselectively modified. For example, in human IgG such as human IgG1, since the following amino acid residues present in the heavy chain constant region may be exposed on the antibody surface, the regioselectivity of the intermediate antibody and the antibody derivative formed in the present invention may be the positions of such amino acids (the positions of amino acid residues rely on EU numbering).

### (1) Exposed Lysine Residues

CH2 domain (e.g., positions 246, 248, 274, 288, 290, 317, 320, and 322)
CH3 domain (e.g., positions 360, 414, and 439)

### (2) Exposed Tyrosine Residues

CH2 domain (e.g., positions 278, 296, and 300)
CH3 domain (e.g., position 436)

### (3) Exposed Serine Residues

CH2 domain (e.g., positions 254, 267, and 298)
CH3 domain (e.g., positions 400, 415, and 440)

### (4) Exposed Threonine Residues

CH2 domain (e.g., positions 256 and 289)
CH3 domain (e.g., positions 335 and 359)

The regioselectivity of the intermediate antibody and the antibody derivative formed in the present invention is preferably the position of the lysine residues or the tyrosine residues in the IgG antibody heavy chain, more preferably the position of the lysine residues in the heavy chain of an IgG antibody, and further more preferably the lysine residue in position 246/248, position 288/290, or position 317 in the heavy chain of an IgG antibody. In the present invention, as long as a specific amino acid residue in the heavy chain of an antibody is regioselectively modified, a different specific amino acid residue in another position may be further modified (regioselectively modified or non-regioselectively modified).

The antibody may be in a free form or may be in a salt form, unless otherwise specified. Examples of the salt include salts with inorganic acids, salts with organic acids, salts with inorganic bases, salts with organic bases, and salt with amino acids. Examples of the salts with inorganic acids include salts with hydrogen chloride, hydrogen bromide, phosphoric acid, sulfuric acid, and nitric acid. Examples of the salts with organic acids include salts with formic acid, acetic acid, trifluoroacetic acid, lactic acid, tartaric acid, fumaric acid, oxalic acid, maleic acid, citric acid, succinic acid, malic acid, benzenesulfonic acid, and p-toluenesulfonic acid. Examples of the salts with inorganic bases include salts with alkali metals (e.g., sodium and potassium), alkaline-earth metals (e.g., calcium and magnesium), other metals such as zinc and aluminum, and ammonium. Examples of the salts with organic bases include salts with trimethylamine, triethylamine, propylenediamine, ethylenediamine, pyridine, ethanolamine, a monoalkyl ethanolamine, a dialkyl ethanolamine, diethanolamine, and triethanolamine. Examples of the salts with amino acids include salts with basic amino acids (e.g., arginine, histidine, lysine, and ornithine) and acidic amino acids (e.g., aspartic acid and glutamic acid). The salt is preferably a salt with an inorganic acid (e.g., hydrogen chloride) or a salt with an organic acid (e.g., trifluoroacetic acid).

The concentration of the antibody material in the solution is not limited as long as the concentration enables a sufficient reaction with the compound included in the reagent for regioselectively modifying an antibody and, for example, may be 0.1 to 30 mg/mL. The concentration may be preferably 0.2 mg/mL or more, more preferably 0.5 mg/mL or more, further more preferably 1.0 mg/mL or more, and particularly preferably 2.0 mg/mL or more. The concentration may be 25 mg/mL or less, 20 mg/mL or less, 18 mg/mL or less, 16 mg/mL or less, or 14 mg/mL or less.

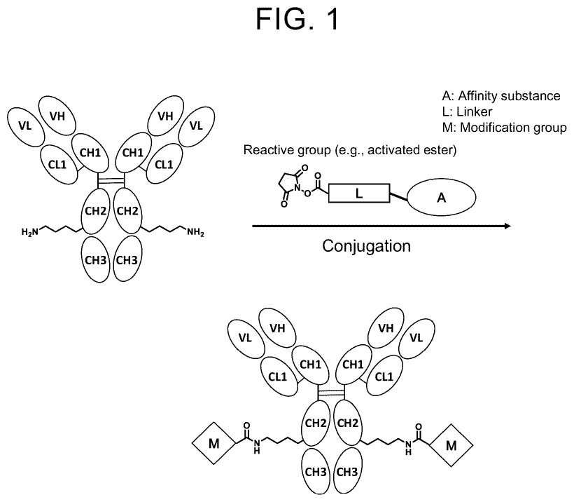

The reagent for regioselectively modifying an antibody used in the process (1) comprises a compound comprising an affinity substance to an antibody and a reactive group to an antibody. With the use of such a compound, an intermediate antibody regioselectively modified can be formed in the process (2) (e.g., see FIGS. 1, 2-1, 3-1, and 4). The compound may be in a free form or may be in a salt form, unless otherwise specified. The salt may be such a salt described above for the antibody.

The affinity substance to an antibody is a substance having affinity to an antibody as described above.

Examples of the affinity substance include peptides [e.g., comprising oligopeptides and polypeptides (proteins), which may be modified with sugar], small compounds, nucleic acids, nucleic acid-peptide conjugates, peptide-small compound conjugates, and nucleic acid-small compound conjugates.

In a specific embodiment, the affinity substance may be a peptide having affinity to a heavy chain constant region of an antibody. As such a peptide, for example, the following peptides have been reported:
(1) an IgG binding peptide having affinity to a specific region (CH2 region) of human IgG in general (that is, human IgG1, IgG2, IgG3, and IgG4. The same applies hereinafter.) (e.g., see WO 2018/199337, WO 2019/240287, WO 2020/090979, WO 2019/240288, WO 2016/186206, WO 2013/027796, and WO 2008/054030, which are incorporated herein by reference in their entireties);
(2) a Protein A Mimetic (PAN) peptide having affinity to a specific region (CH2 region) of human IgG in general (e.g., see Fassina G et al., JOURNAL OF MOLECULAR RECOGNITION, 1996, VOL. 6, 564-569, which is incorporated herein by reference in its entirety);
(3) EPIHRSTLTALL (SEQ ID NO: 1) having affinity to a specific region (CH2 region) of human IgG in general (e.g., see Ehrlich G. K et al., J. Biochem. Biophys. Methods, 2001, VOL. 49, 443-454, which is incorporated herein by reference in its entirety);
(4) (NH₂-Cys1-X1-X2-X3-X4)₂-Lys-Gly-OH having affinity to a specific region (Fc region) of human IgG in general (e.g., see Ruvo M et al., ChemBioChem, 2005, VOL. 6, 1242-1253, which is incorporated herein by reference in its entirety);
(5) FARLVSSIRY (SEQ ID NO: 2), FGRLVSSIRY (SEQ ID NO: 3), and TWKTSRISIF (SEQ ID NO: 4) having affinity to a specific region (Fc region) of human IgG in general (e.g., see Krook M et al., Journal of Immunological Methods, 1998, VOL. 221, 151-157, which is incorporated herein by reference in its entirety);
(6) QSYP (SEQ ID NO: 5) having affinity to a specific region of human IgG in general (e.g., see Jacobs J. M. et al., Bio. Techniques, 2003, VOL. 34, 132-141, which is incorporated herein by reference in its entirety);
(7) HWRGWV (SEQ ID NO: 6), HYFKFD (SEQ ID NO: 7), and HFRRHL (SEQ ID NO: 8) having affinity to a specific region (Fc region) of human IgG in general (e.g., see Carbonell R. G. et al., Journal of Chromatography A, 2009, VOL. 1216, 910-918, which is incorporated herein by reference in its entirety);
(8) DAAG (SEQ ID NO: 9) having affinity to a specific region (Fc region) of human IgG in general (e.g., see Lund L. N. et al., Journal of Chromatography A, 2012, VOL. 1225, 158-167, which is incorporated herein by reference in its entirety);
(9) Fc-I, Fc-II, and Fc-III having affinity to a specific region (Fc region) of human IgG in general (e.g., see Warren L. Delano et al., Science, 2000, VOL. 287, 1279-1283; WO 2001/045746, which are incorporated herein by reference in their entireties); and
(10) NARKFYKG (SEQ ID NO: 10) and NKFRGKYK (SEQ ID NO: 11) having affinity to a specific region (Fc region) of human IgG in general (e.g., see Biochemical Engineering Journal, 2013, VOL. 79, 33-40, which is incorporated herein by reference in its entirety).

In another specific embodiment, the affinity substance may be a substance other than the peptide. Reported examples of such a substance include an aptamer having affinity to a specific region (the CH2 region, especially a side chain of Lys340) of human IgG in general [e.g., GGUG(C/A) (U/T) motif-containing aptamers such as GGUGCU and GGUGAU] (e.g., see WO 2007/004748; Nomura Y et al., Nucleic Acids Res., 2010 November; 38(21): 7822-9; Miyakawa S et al., RNA., 2008 June; 14(6): 1154-63, which are incorporated herein by reference in their entireties).

The affinity substance described above can be obtained by any known method in the art. For example, the affinity substance can be obtained by producing an antibody (e.g., the hybridoma method) using the entire antibody or a target portion in the antibody or screening the affinity substance (e.g., the phage display method, the SELEX method, the mRNA display method, the ribosome display method, the cDNA display method, and the yeast display method) from a library from which the affinity substance is available (e.g., peptide libraries, antibody libraries, antibody-forming cell libraries, aptamer libraries, phage libraries, mRNA libraries, and cDNA libraries). When the affinity substance to an antibody is an affinity substance to the Fc region (a soluble region) of an antibody, a partial peptide present in a specific region (e.g., CH1, CH2, and CH3) of the Fc region of various kinds of antibodies (e.g., IgG, IgA, IgM, IgD, and IgE) is used, whereby an affinity substance capable of selectively binding to any portion in the Fc region of the antibody can be efficiently obtained. The thus obtained affinity substances comprise a mixture of substances relatively strong and weak in affinitive binding ability. However, even an affinity substance weak in affinitive biding ability can strengthen its affinitive binding ability when used in an excessive amount.

In a preferred embodiment, the affinity substance may be a peptide identified as having ability to be affinitively associated with a heavy chain (preferably CH2 region) of an antibody (preferably IgG antibody) and enabling regioselective modification to a lysine residue (e.g., the lysine residue in position 246/248, position 288/290, or position 317 in the IgG antibody) in the heavy chain (preferably CH2 region) of the antibody. Examples of the peptide include a variety of peptides described in WO 2016/186206, WO 2018/199337, WO 2019/240287, WO 2020/090979, and WO 2019/240288, which are incorporated herein by reference in their entireties.

The reactive group to an antibody is a group that enables a reaction to an amino acid residue present in an antibody that is a kind of proteins. Proteins are normally composed of 20 kinds of natural amino acids. The amino acids are alanine (A), asparagine (N), cysteine (C), glutamine (Q), glycine (G), isoleucine (I), leucine (L), methionine (M), phenylalanine (F), proline (P), serine (S), threonine (T), tryptophan (W), tyrosine (Y), valine (V), aspartic acid (D), glutamic acid (E), arginine (R), histidine (H), and lysine (K). Among those amino acids, glycine with no side chain, and alanine, isoleucine, leucine, phenylalanine, and valine of which side chains are hydrocarbon groups are inactive to a normal reaction. The reactive group to an antibody is therefore a group capable of reacting to a functional group in the side chain of one or two or more kinds (e.g., two kinds, three kinds, or four kinds) of 14 kinds of amino acids consisting of asparagine, glutamine, methionine, proline, serine, threonine, tryptophan, tyrosine, aspartic acid, glutamic acid, arginine, histidine, and lysine. One kind or two or more kinds (e.g., two kinds, three kinds, or four kinds) of reactive groups may be included in the compound. In terms of simplification or the like of the compound, only one kind of reactive group may be included in the compound.

In a specific embodiment, the reactive group may be a reactive group to a functional group (that is, an amino group or a hydroxy group) in a side chain of one kind of amino acids of lysine, tyrosine, serine, and threonine (preferably, lysine or tyrosine). For example, in human IgG such as human IgG1, since the amino acid residues as described above present in the heavy chain constant region may be exposed on the antibody surface, these amino acids can be used as a reaction target of the reactive group.

In a preferred embodiment, the reactive group may be a reactive group to an amino group that is a functional group specific to a side chain of lysine. Examples of such a reactive group include an activated ester group (e.g., an N-hydroxysuccinimide group), a vinylsulfone group, a sulfonylchloride group, an isocyanate group, an isothiocyanate group, an imidazolylcarbonyl group, a carbonate group, an aldehyde group, a 1,4,7,10-tetraazacyclododecane-1,4,7,10-tetraacetic acid group, a 2-imino-2-methoxyethyl group, and a diazonium terephthalic acid group.

In a specific embodiment, the reactive group may be any electrophilic group capable of coupling with a leaving group described later and having ability to react with a nucleophilic group (e.g., NH₂ in a side chain of a lysine residue, and a hydroxy group in a side chain of a tyrosine residue, a serine residue, and/or a threonine residue) in the antibody and preferably may be a group selecting from the group consisting of —C(═O)—, —SO₂—, and —CH₂— (e.g., WO 2019/240288, which is incorporated herein by reference in its entirety). As the electrophilic group, —CH₂— can also be used depending on an electronic balance with a group coupling therewith (e.g., a leaving group). For example, when a tosyl group is used as the leaving group, —CH₂— can be suitably used as the electrophilic group (Tsukiji et al., Nature Chemical Biology, Vol. 5, No. 5, May 2009, which is incorporated herein by reference in its entirety). The electrophilic group is more preferably —C(═O)— or —SO₂— and further more preferably —C(═O)—.

In a specific embodiment, the compound comprising an affinity substance and a reactive group may further comprise one or more portions selected from the group consisting of a bioorthogonal functional group, a cleavable site, and a leaving group. The compound may comprise such a portion at any position. Preferably, the compound may comprise such a portion at a position between the affinity substance and the reactive group.

The bioorthogonal functional group refers to a group that does not react with biological components (e.g., amino acids, proteins, nucleic acids, lipids, sugars, and phosphoric acid) or has a slow reaction to the biological components but selectively reacts with components other than the biological components. The bioorthogonal functional group is well known in the art (e.g., see Sharpless K. B. et al., Angew. Chem. Int. Ed. 40, 2004 (2015); Bertozzi C. R. et al., Science 291, 2357 (2001); Bertozzi C. R. et al., Nature Chemical Biology 1, 13 (2005), which are incorporated herein by reference in their entireties).

In the present invention, a bioorthogonal functional group to proteins is used as the bioorthogonal functional group. This is because the antibody to be modified is a protein. The bioorthogonal functional group to proteins is a group that does not react with side chains of 20 kinds of natural amino acid residues normally forming proteins or has a slow reaction to the side chains but can react with a target functional group. Among those amino acids, glycine with no side chain, and alanine, isoleucine, leucine, phenylalanine, and valine of which side chains are hydrocarbon groups are inactive to a normal reaction. The bioorthogonal functional group to proteins is therefore a group that does not react with, in addition to the side chains of these amino acids having side chains inactive to normal reactions, side chains of asparagine, glutamine, methionine, proline, serine, threonine, tryptophan, tyrosine, aspartic acid, glutamic acid, arginine, histidine, and lysin, or has a slow reaction therewith but reacts with a target functional group. As the bioorthogonal functional group, a group different from the reactive group described above can be used. For example, when a reactive group to an amino group that is a functional group in a side chain of lysine is used, the bioorthogonal functional group is a group that does not react or has a slow reaction with an amino group but reacts with a target functional group other than an amino group.

Examples of the bioorthogonal functional group to proteins include an azide residue, an aldehyde residue, an alkene residue (in other words, it is only required to have a vinylene (ethenylene) portion, which is a minimum unit having a carbon-carbon double bond), an alkyne residue (in other words, it is only required to have an ethynylene portion, which is a minimum unit having a carbon-carbon triple bond), a halogen residue, a tetrazine residue, a nitrone residue, a hydroxylamine residue, a nitrile residue, a hydrazine residue, a ketone residue, a boronic acid residue, a cyanobenzothiazole residue, an allyl residue, a phosphine residue, a maleimide residue, a disulfide residue, a thioester residue, an α-halocarbonyl residue (e.g., a carbonyl residue having a fluorine atom, a chlorine atom, a bromine atom, or an iodine atom at an α-position. The same applies hereinafter), an isonitrile residue, a sydnone residue, and a selenium residue.

In the present invention, since the bioorthogonal functional group to an antibody in which all thiol groups in a side chain of a cysteine residue are subjected to disulfide bonds can be used, a thiol residue can be used as the bioorthogonal functional group, in addition to normal bioorthogonal functional groups to proteins.

Preferably, the bioorthogonal functional group may be a group selected from the group consisting of an azide residue, a thiol residue, an alkyne residue, and a maleimide residue, among the bioorthogonal functional groups described above, in terms of improving reaction efficiency and the like.

The cleavable site is a site that can be cleaved by a specific process. In the present invention, the cleavable site is preferably a site that can be cleaved by a specific process under a condition (mild condition) that cannot induce denaturation or degradation of proteins (e.g., cleavage of amide bonds). It is therefore can be said that the cleavable site is a site (a bond other than an amide bond forming a protein) that can be cleaved by a specific cleavage process under a mild condition. Examples of the specific process include (a) a process by a cleavage agent as described later, (b) a process by physiochemical stimulation (e.g., light), and (c) leaving to stand when an autolytic cleavable site is used. Such cleavable sites and cleavage conditions thereof are common technical knowledge in the art (e.g., G. Leriche, L. Chisholm, A. Wagner, Bioorganic & Medicinal Chemistry. 20, 571 (2012); Feng P. et al., Journal of American Chemical Society. 132, 1500 (2010); Bessodes M. et al., Journal of Controlled Release, 99, 423 (2004); DeSimone, J. M., Journal of American Chemical Society. 132, 17928 (2010); Thompson, D. H., Journal of Controlled Release, 91, 187 (2003); Schoenmarks, R. G., Journal of Controlled Release, 95, 291 (2004), which are incorporated herein by reference in their entireties). Preferably, the cleavable site is a site that can be cleaved by a process with a cleavage agent. Examples of such a cleavable site include a disulfide residue, an acetal residue, a ketal residue, an ester residue, a carbamoyl residue, an alkoxyalkyl residue, an imine residue, a tertiary alkyloxy carbamate residue (e.g., a tert-butyloxy carbamate residue), a silane residue, a hydrazone-containing residue (e.g., a hydrazone residue, an acyl hydrazone residue, and a bisaryl hydrazone residue), a phosphoramidate residue, an aconityl residue, a trityl residue, an azo residue, a vicinal diol residue, a selenium residue, an aromatic ring-containing residue having an electron-withdrawing group, a coumarin-containing residue, a sulfone-containing residue, an unsaturated bond-containing chain residue, and a glycosyl residue.

Examples of the electron-withdrawing group include a halogen atom, halogen atom-substituted alkyl (e.g., trifluoromethyl), a boronic acid residue, mesyl, tosyl, triflate, nitro, cyano, a phenyl group, and a keto group (e.g., acyl).

In a specific embodiment, the cleavable site may be a cleavable site capable of forming a bioorthogonal functional group by cleavage. Preferably, the cleavable site capable of forming a bioorthogonal functional group by cleavage is a cleavable site that is present between the affinity substance and the reactive group and can form a bioorthogonal functional group on the antibody side by cleavage. Examples of such a cleavable portion include a disulfide residue, a thioester residue, an acetal residue, a ketal residue, an imine residue, and a vicinal diol residue. Examples of the combination of the cleavable site capable of forming a bioorthogonal functional group by cleavage and the bioorthogonal functional group are as follows.

TABLE A
Examples of the combination of the cleavable site capable
of forming a bioorthogonal functional group by cleavage
and the bioorthogonal functional group

| | Bioorthogonal functional |
|---|---|
| Cleavable site | group |
| Disulfide residue | Thiol residue |
| Thioester residue | Thiol residue |
| Acetal residue | Aldehyde residue |
| Ketal residue | Ketone residue |
| Imine residue | Ketone residue |
| Vicinal diol residue | Aldehyde residue |

In a specific embodiment, the cleavable site capable of forming a bioorthogonal functional group by cleavage may be a disulfide residue or a thioester residue capable of forming a thiol group by cleavage.

The leaving group is a group having ability to be cleaved and eliminated by a reaction between a nucleophilic group in the antibody and the reactive group (electrophilic group) in the compound. In the present invention, the leaving group is a group having ability to be eliminated by a specific process that cannot induce denaturation or degradation of proteins (e.g., cleavage of amide bonds). Such a leaving group is common technical knowledge in the art (e.g., WO 2019/240288; Fujishima, S. et al. J. Am. Chem. Soc., 2012, 134, 3961-3964. (described above); Chem. Sci. 2015 3217-3224.; Nature Chemistry volume 8, pages 542-548 (2016), which are incorporated herein by reference in their entireties). Examples of such a leaving group include (1) a group selected from the group consisting of —O—, —S—, —Se—, —SO₂—O—, —SO₂—N(R)—, —SO₂—, —C≡C—CH₂—O—, —N(OR)—, —N(R)—, and —O—N(R)— (wherein R is a hydrogen atom or alkyl having 1 to 6 carbon atoms), and (2) heteroarylene.

Examples of alkyl having 1 to 6 carbon atoms include methyl, ethyl, n-propyl, i-propyl, n-butyl, s-butyl, isobutyl, t-butyl, pentyl, and hexyl. Alkyl having 1 to 6 carbon atoms is preferably alkyl having 1 to 4 carbon atoms.

The heteroarylene used as the leaving group is a heteroarylene having a low n electron density (that is, less than 1). The heteroarylene is preferably a heteroarylene containing a nitrogen atom as a ring-constituting atom. The heteroarylene containing a nitrogen atom as a ring-constituting atom is preferably a heteroarylene having 1 to 21 carbon atoms and containing a nitrogen atom as a ring-constituting atom, more preferably a heteroarylene having 1 to 15 carbon atoms and containing a nitrogen atom as a ring-constituting atom, and even more preferably a heteroarylene having 1 to 9 carbon atoms and containing a nitrogen atom as a ring-constituting atom. The heteroarylene, which is a leaving group, may be substituted with or is not necessarily substituted with a substituent such as an electron-withdrawing group as described above. The number of carbon atoms does not comprise the number of carbon atoms of the substituent. Examples of such a heteroarylene include imidazolediyl, triazolediyl, tetrazolediyl, and 2-pyridonediyl (that is, 2-hydroxypyridinediyl).

In a preferred embodiment, the compound may be a first specific compound comprising an affinity substance to an antibody, a reactive group to an antibody, and a cleavable site and optionally further comprising a bioorthogonal functional group.

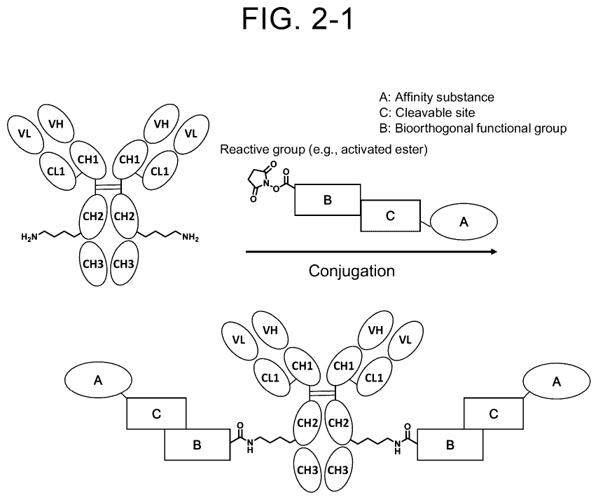

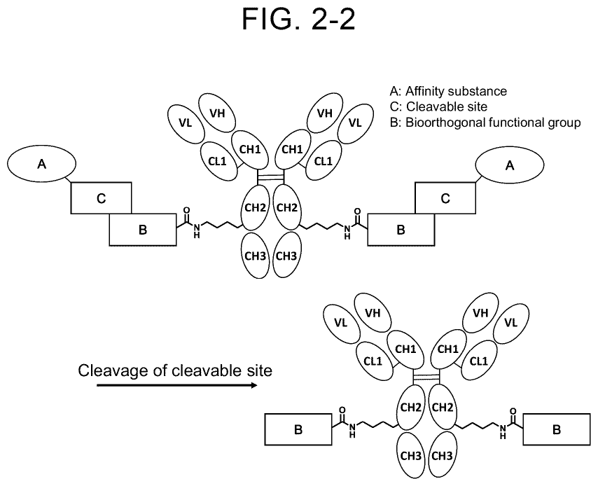

A preferable example of the first specific compound is a compound comprising an affinity substance to an antibody, a reactive group to an antibody, a cleavable site, and a bioorthogonal functional group. In this case, (i) the cleavable site may be present at an affinity substance-side position between the affinity substance and the reactive group, or (ii) the bioorthogonal functional group may be present at a reactive group-side position between the affinity substance and the reactive group. That is, the cleavable site may be present at a position relatively closer to the affinity substance than the bioorthogonal functional group. With the use of such a compound, in the process (2), an intermediate antibody regioselectively having a specific structural unit comprising an affinity substance, a cleavable site, and a bioorthogonal functional group can be formed (FIG. 2-1).

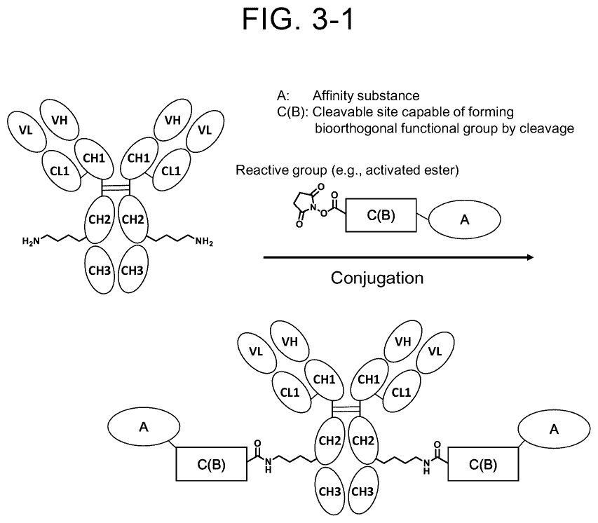

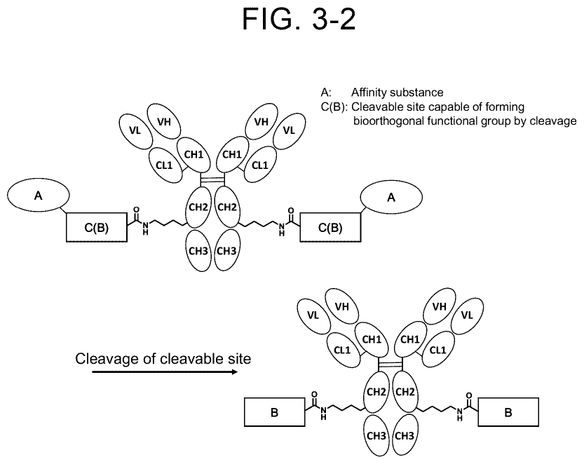

Another preferable example of the first specific compound is a compound comprising an affinity substance to an antibody, a reactive group to an antibody, and a cleavable site capable of forming a bioorthogonal functional group by cleavage. In this case, the cleavable site capable of forming a bioorthogonal functional group by cleavage may be present at a position between the affinity substance to an antibody and the reactive group to an antibody. With the use of such a compound, in the process (2), an intermediate antibody regioselectively having a specific structural unit comprising an affinity substance and a cleavable site capable of forming a bioorthogonal functional group by cleavage can be formed (FIG. 3-1).

In a specific embodiment, the first specific compound may be a compound represented by the following Formula (I):

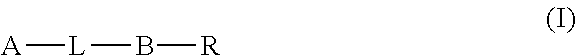

wherein
A is an affinity substance to a soluble protein,
L is a cleavable linker that is a divalent group comprising a cleavable portion,
B is (a) a divalent group comprising a bioorthogonal functional group or (b) a divalent group not comprising a bioorthogonal functional group, and
R is a reactive group to the soluble protein (e.g., WO 2018/199337, WO 2019/240287, and WO 2020/090979, which are incorporated herein by reference in their entireties).

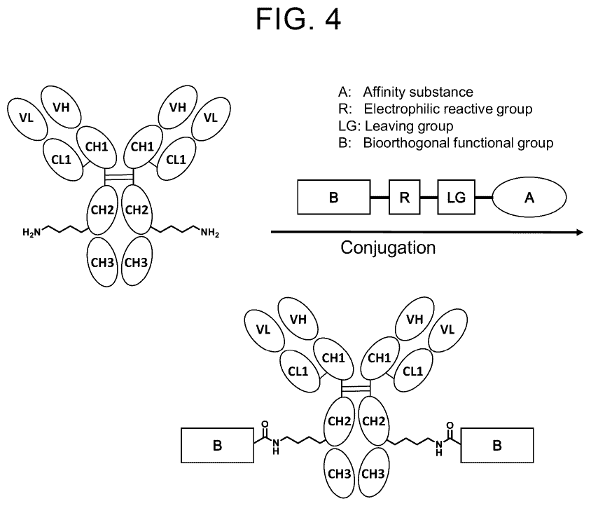

In another preferred embodiment, the compound may be a second specific compound comprising an affinity substance to an antibody, a reactive group to an antibody (electrophilic group), a leaving group, and a bioorthogonal functional group. In this case, (i) the leaving group and the reactive group may be coupled with each other and present between the affinity substance and the bioorthogonal functional group, (ii) the leaving group may be present at an affinity substance-side position between the affinity substance and the bioorthogonal functional group, and (iii) the reactive group may be present at a bioorthogonal functional group-side position between the affinity substance and the bioorthogonal functional group. That is, the leaving group may be present at a position relatively closer to the affinity substance than the reactive group. With the use of such a compound, in the process (2), an intermediate antibody regioselectively having a bioorthogonal functional group or bioorthogonal functional groups can be formed (FIG. 4).

In a specific embodiment, the second specific compound may be a compound represented by the following Formula (II):

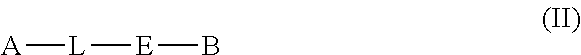

wherein
A is an affinity substance to an antibody,
L is a divalent group comprising a leaving group,
E is a divalent group comprising an electrophilic group (i) coupled with the leaving group and (ii) having ability to react with a nucleophilic group in the antibody,
B is a bioorthogonal functional group, and
the leaving group has ability to be cleaved and eliminated from E by a reaction between the nucleophilic group and the electrophilic group (e.g., WO 2019/240288, which is incorporated herein by reference in its entirety).

The concentration of the compound in the solution is not limited as long as the concentration enables a sufficient reaction with the antibody. For example, the concentration may be 0.05 to 30 mM. The concentration may be preferably 0.1 mM or more, more preferably 0.2 mM or more, further more preferably 0.3 mM or more, and particularly preferably 0.4 mM or more. The concentration may be 20 mM or less, 10 mM or less, 5 mM or less, 2 mM or less, or 1 mM or less. The concentration may be defined as an equivalent to the antibody. Thus, such a concentration may be, for example, 1 to 100 molar equivalents, preferably 1 to 50 molar equivalents (or 2 to 50 molar equivalents), more preferably 1 to 30 molar equivalents (or 3 to 30 molar equivalents), further more preferably 1 to 20 molar equivalents (or 4 to 20 molar equivalents), and particularly preferably 1 to 15 molar equivalents (or 5 to 15 molar equivalents) to the antibody.

In the present invention, an aqueous solution can be used as a solution such as the solution comprising an antibody material and the solution comprising a reagent for regioselectively modifying an antibody. Examples of the aqueous solution include water (e.g., distilled water, sterile distilled water, purified water, and physiological saline) and buffer solutions (e.g., aqueous solutions of phosphoric acid, Tris-hydrochloric buffer solution, carbonic acid-bicarbonate buffer solution, aqueous solution of boric acid, glycine-sodium hydroxide buffer solution, and citric acid buffer solution), and buffer solutions are preferred. The pH of the solution is, for example, 5.0 to 9.0 and preferably 5.5 to 8.5. The aqueous solution may comprise other components. The other components are, for example, any components such as chelators, organic solvents (e.g., alcohols), and salts.

The first and second inlet channels as described above can be designed to be identical or different channels. For example, the length, the representative diameter, the shape, and the material of the first and second inlet channels are not limited as long as each of the solution comprising an antibody material and the solution comprising the reagent can be introduced to the junction of the first inlet channel and the second inlet channel. The length of the first and second inlet channels is, for example, 0.1 to 10 meters, preferably 0.1 to 5 meters, and more preferably 0.2 to 3 meters. The representative diameter of the first and second inlet channels is, for example, 0.1 to 3.0 mm, preferably 0.2 to 2.5 mm, and more preferably 0.4 to 2.0 mm. As used herein “representative diameter” refers to the diameter of a cylinder equivalent to the area of the channel section. Thus, when the shape of the channel section is circular, the representative diameter is the inner diameter. On the other hand, when the shape of the channel section is non-circular with identical or different width and depth, the representative diameter is the diameter of a cylinder having a section equivalent to the section obtained from the width and the depth. The shape of the first and second inlet channels may be linear or nonlinear [e.g., a shape having one or more curved portions and linear portions, a circular shape (e.g., a coil-like shape and a spiral shape)]. Examples of the material of the first and second inlet channels include metal materials [e.g., stainless steel materials (SUS), Hastelloy (registered trademark), and Inconel], resins [e.g., polytetrafluoroethylene (PTFE), polyethersulfone (PES), polyether ether ketone (PEEK), polydimethylsiloxane (PDMS), and tetrafluoroethylene-perfluoroalkyl vinyl ether copolymer (PFA)], and glass.

The flow rates of the solutions in the first inlet channel and the second inlet channel may be identical or different and, for example, may be 0.1 to 20 mL/min. The flow rate may be preferably 0.2 mL/min or more, more preferably 0.3 mL/min or more, further more preferably 0.4 mL/min or more, and particularly preferably 0.5 mL/min or more. The flow rate may be 15 mL/min or less, 10 mL/min or less, 5 mL/min or less, or 2 mL/min or less. More specifically, the flow rate may be preferably 0.2 to 15 mL/min, more preferably 0.3 to 10 mL/min, further more preferably 0.4 to 5 mL/min, and particularly preferably 0.5 to 2 mL/min.

At the junction of the first inlet channel and the second inlet channel, any given micromixer can be used. Examples of the micromixer include micromixers of a variety of mixing types, such as a collision type (e.g., T-shaped), a sheath flow type, a static type, a helix type, and a multilayer flow type. The representative diameter of the micromixer at a junction of the first inlet channel and the second inlet channel is preferably equal to or smaller than the representative diameter of the first inlet channel and/or the second inlet channel. The representative diameter of such a micromixer may be, for example, 1.0 mm or less, 0.9 mm or less, 0.8 mm or less, 0.7 mm or less, 0.6 mm or less, 0.5 mm or less, 0.4 mm or less, 0.3 mm or less, or 0.25 mm or less. The representative diameter of the micromixer may be 0.05 mm or more or 0.1 mm or more. The shape of the channel section of the junction in the micromixer may be non-circular with identical or different width and depth or may be circular. A single junction of the first inlet channel and the second inlet channel may be provided, or multiple junctions (when there are a plurality of first inlet channels and/or second inlet channels) may be provided. In view of easy designing and manufacturing of FMRs, a single junction is preferable. Examples of the material of the micromixer include metal materials [e.g., stainless steel materials (SUS), Hastelloy (registered trademark), and Inconel], resins [e.g., polytetrafluoroethylene (PTFE), polyethersulfone (PES), polyether ether ketone (PEEK), polydimethylsiloxane (PDMS), and tetrafluoroethylene-perfluoroalkyl vinyl ether copolymer (PFA)], and glass.

In a specific embodiment, each of the representative diameter ratio of the micromixer to the first inlet channel (micromixer/first inlet channel) and the representative diameter ratio of the micromixer/the second inlet channel (micromixer/second inlet channel) may be less than 1.0. With such a representative diameter ratio, the solution is accelerated at the junction to produce more minute solution units, and therefore a homogeneous solution can be formed more rapidly. Such a representative diameter ratio may be, for example, 0.95 or less, 0.90 or less, 0.85 or less, 0.80 or less, 0.75 or less, 0.70 or less, 0.65 or less, 0.60 or less, 0.55 or less, 0.50 or less, 0.45 or less, 0.40 or less, 0.35 or less, 0.30 or less, or 0.25 or less. The smaller the representative diameter ratio is, the more rapidly a homogeneous solution can be formed. Such a representative diameter ratio may be 0.05 or more, or 0.1 or more.

In a specific embodiment, the micromixer may be a collision-type micromixer. In the present invention, the collision-type micromixer refers to a micromixer that promotes the mixing by producing a mixing vortex at the time of contact between a plurality of solutions. In the present invention, a micromixer used as the collision-type micromixer is provided with a junction into which a plurality of solutions flow from a plurality of inlet channels including at least two inlet channels disposed in a positional relation that enables production of a mixing vortex at the time of contact between a first solution and a second solution containing different components that react with each other. As used herein, the positional relation refers to a relation in which the two inlet channels (the direction in which the solution flows in) are at facing positions or at positions inclined to the outlet channel at an angle (angles X and Y, respectively) relative to the facing position (Table B). In the two inlet channels, the angle X set for the first inlet channel (e.g., at least one first reaction channel) is an angle inclined to the outlet side relative to the facing position, and the angle Y set for the second inlet channel (e.g., at least one third inlet channel) is an angle inclined to the outlet channel side relative to the facing position. In such a case, two solutions in at least two inlet channels (the first inlet channel and the second inlet channel) can collide in flows in directions completely or substantially opposite to each other to significantly increase the collision force. Even when the collision of flows in directions completely opposite each other does not occur, the solutions collide against the channel wall (solid phase) in the micromixer and do not lose the collision force, causing a strong collision. The angles X and Y may be identical or may be different, within 30°, preferably within 25°, more preferably within 20°, further more preferably within 15°, particularly preferably within 10°, within 9°, within 8°, within 7°, within 6°, within 5°, within 4°, within 3°, within 2°, or within 1°. The smaller the angle is, the stronger the collision force of the solutions from two inlet channels is, and a homogeneous solution can be generated more rapidly. Such a collision-type micromixer used in the present invention differs from a static-type micromixer which promotes mixing with a structure in the channel after a plurality of the solutions merge. The collision-type micromixer used in the present invention differs from a sheath flow-type micromixer in which both of the first solution and the second solution containing different components that should react with each other flow into the micromixer in a positional relation in a forward direction and flow out to the outlet channel, and thus tend to lose the collision force (that is, fail to collide strongly) (for example, the sheath flow-type micromixer described in WO2017/179353, which is incorporated herein by reference in its entirety, in which at least one incoming solution flows into the micromixer in the forward direction to the outgoing solution). Preferably, the collision-type micromixer is a T-shaped micromixer including two inlet channels facing each other to pass the first solution and the second solution containing different components that should react with each other (e.g., the first reaction channel allowing the solution containing an antibody derivative having a specific reactive site and a cleavage agent to pass through, and the third inlet channel allowing a solution containing a drug to pass through), and including a junction channel at which two inlet channels and one outlet channel are orthogonal to each other.

TABLE B
Relation between at least two inlet channels and
angles X and Y

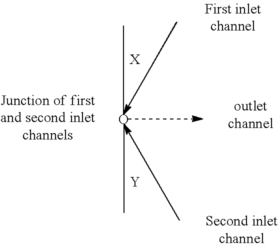

Examples of the relation between at least two inlet channels
(first and second inlet channels)

| | | | |
|---|---|---|---|
| 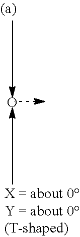 | 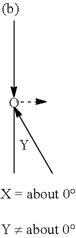 | 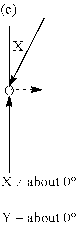 | 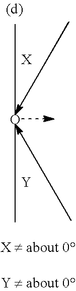 |

In (a), the microreactor in which the outlet channel is on the same plane as the first inlet channel and the second inlet channel is a T-shaped microreactor.

### 1-3. Process (2)

In the process (2), the mixture obtained in the process (1) can be passed through a reaction channel to allow the antibody material and the reagent to react in the reaction channel to form a solution comprising the intermediate antibody regioselectively modified. For example, when the mixture formed as described above is passed through the reaction channel, the antibody material and the compound included in the reagent react in the reaction channel. A solution comprising the intermediate antibody regioselectively modified is thus formed.

The reaction channel can be designed such that a desired residence time of the mixture in the reaction channel can be achieved in order to control the reaction time in the reaction between the antibody material and the compound. Such a residence time is not specifically limited, but may be, for example, less than 15 minutes, preferably less than 10 minutes, more preferably less than 8 minutes, and further more preferably less than 6 minutes. The residence time of the mixture in the reaction channel can be controlled, for example, by adjusting factors such as the flow rates of the solutions in the first inlet channel and the second inlet channel and the length of the reaction channel and the representative diameters of the channels.

The reaction temperature in the reaction channel can be readily controlled. In an FMR with a large surface area per unit volume, heat transfer occurs fast and the temperature therefore can be controlled precisely and rapidly. The reaction temperature can be controlled, for example, using a temperature controller attached to the outside of the reaction channel or using a bath in which the reaction channel or the micromixer arranged upstream thereof can be immersed (e.g., water bath), or using a pre-temperature adjustment mechanism (e.g., coil residence tube). The reaction in the reaction channel can be performed under a mild condition described later.

The reaction channel is not limited as long as it is constructed such that the residence time of the mixture as described above can be achieved, and, for example, the length, the representative diameter, the shape, and the material of the reaction channel may be set as follows. The length of the reaction channel may be, for example, 1 to 30 meters, preferably 2 to 20 meters, more preferably 3 to 15 meters, and further more preferably 4 to 10 meters. The representative diameter of the reaction channel may be, for example, 0.5 to 3 mm, preferably 0.6 to 2.5 mm, more preferably 0.7 to 2.0 mm, and further more preferably 0.8 to 1.5 mm. The shape of the reaction channel may be linear or nonlinear [e.g., a shape having one or more curved portions and linear portions, and a circular shape (e.g., coil-like shape and spiral shape)]. A material similar to the first and second inlet channels can be used as the material of the reaction channel.

The reaction in the reaction channel can be performed under a mild condition that cannot induce denaturation or degradation of the antibody (e.g., cleavage of amide bonds) (for example, see G. J. L. Bernardes et al., Chem. Rev., 115, 2174 (2015); G. J. L. Bernardes et al., Chem. Asian. J., 4, 630 (2009); B. G. Davies et al., Nat. Commun., 5, 4740 (2014); A. Wagner et al., Bioconjugate. Chem., 25, 825 (2014), which are incorporated herein by reference in their entireties). The reaction temperature under a mild condition may be, for example, 4 to 50° C., preferably 10 to 40° C., and more preferably room temperature (e.g., 15 to 30° C.). The degree of reaction can be controlled, for example, by adjusting factors such as the concentrations of the antibody material and the compound, the residence time of the mixture in the reaction channel, and the reaction temperature in the reaction channel.

In a specific embodiment, the time taken for formation of the intermediate antibody regioselectively modified can be defined by the time required from arrival at the given micromixer to passage through the reaction channel of the solution comprising an antibody material and the solution comprising the compound. Given that the mixing by the micromixer can be performed instantaneously, the time required for formation of the intermediate antibody regioselectively modified can be mainly determined according to the residence time of the mixture in the reaction channel. Preferably, the residence time of the mixture in the reaction channel may be controlled within 3 minutes. For example, such control can be achieved by adjusting factors such as the flow rates of the solutions in the first inlet channel and the second inlet channel and the length and the representative diameter of the reaction channel. Preferably, the residence time of the mixture in the reaction channel may be within 2.5 minutes, within 2 minutes, within 1.5 minutes, within 1 minute, within 50 seconds, within 40 seconds, within 30 seconds, within 20 seconds, or within 10 seconds. It was demonstrated that the method described in Examples can achieve conjugation performed by the processes (1) and (2) even in an extremely short time as short as 1 second.

### 2. Method for Producing Antibody Derivative Regioselectively Having Bioorthogonal Functional Group

### 2-1. Overview

The present invention provides a method for producing an antibody derivative regioselectively having a bioorthogonal functional group or bioorthogonal functional groups. The method according to the present invention includes the following (1) to (4):
(1) mixing a solution comprising an antibody material and a solution comprising a reagent for regioselectively modifying an antibody in a first micromixer to form a first mixture comprising the antibody material and the reagent,
wherein the reagent comprises a compound comprising an affinity substance to an antibody, a reactive group to an antibody, and a cleavable site and optionally further comprising a bioorthogonal functional group;
(2) passing the first mixture through a first reaction channel to allow the antibody material and the reagent to react in the first reaction channel to form a solution comprising an intermediate antibody regioselectively modified,
wherein the intermediate antibody regioselectively modified is an intermediate antibody regioselectively having a structural unit comprising the affinity substance and the cleavable site and optionally further comprising a bioorthogonal functional group;
(3) mixing the solution comprising the intermediate antibody and a solution comprising a cleavage agent in a second micromixer to form a second mixture comprising the intermediate antibody and the cleavage agent; and
(4) passing the second mixture through a second reaction channel to allow the intermediate antibody and the cleavage agent to react in the second reaction channel to form a solution comprising an antibody derivative regioselectively having a bioorthogonal functional group or bioorthogonal functional groups,
wherein the bioorthogonal functional group in the antibody derivative is (a) the bioorthogonal functional group in the compound or (b) a bioorthogonal functional group formed by cleavage of the cleavable site by the cleavage agent.

The processes (1) to (4) above are continuously performed in a flow microreactor.

A preferable example of the compound is a compound comprising an affinity substance, a reactive group, a cleavable site, and a bioorthogonal functional group. In this case, (i) the cleavable site may be present at an affinity substance-side position between the affinity substance and the reactive group, and (ii) the bioorthogonal functional group may be present at a reactive group-side position between the affinity substance and the reactive group. That is, the cleavable site may be present at a position relatively closer to the affinity substance than the bioorthogonal functional group. With the use of such a compound, in the process (2), an intermediate antibody regioselectively having a specific structural unit comprising an affinity substance, a cleavable site, and a bioorthogonal functional group can be formed (FIG. 2-1), and in the process (4), an antibody derivative regioselectively having a bioorthogonal functional group or bioorthogonal functional groups can be formed (FIG. 2-2).

Another preferable example of the compound is a compound comprising an affinity substance, a reactive group, and a cleavable site capable of forming a bioorthogonal functional group by cleavage. Such a compound may further comprise or does not necessarily comprise a bioorthogonal functional group. This is because even when a bioorthogonal functional group is not further included, an antibody derivative regioselectively having a bioorthogonal functional group or bioorthogonal functional groups can be produced since a bioorthogonal functional group can be formed by cleavage of the cleavable site by a cleavage agent. In this case, the cleavable site capable of forming a bioorthogonal functional group by cleavage may be present at a position between the affinity substance to an antibody and the reactive group to an antibody. With the use of such a compound, in the process (2), an intermediate antibody regioselectively having a specific structural unit comprising an affinity substance and a cleavable site capable of forming a bioorthogonal functional group by cleavage can be formed (FIG. 3-1), and in the process (4), an antibody derivative regioselectively having a bioorthogonal functional group or bioorthogonal functional groups can be formed (FIG. 3-2).

### 2-2. Process (1)

The process (1) in the method for producing an antibody derivative regioselectively having a bioorthogonal functional group or bioorthogonal functional groups can be performed in the same manner as the process (1) in the method for producing an intermediate antibody regioselectively modified. Therefore, the definitions, examples, and preferable examples of terms such as antibody material, reagent for regioselectively modifying an antibody, compound, solution, micromixer (first micromixer), mixing, mixture (first mixture), affinity substance to an antibody, reactive group to an antibody, cleavable site, and bioorthogonal functional group, and the detail of embodiments such as conditions of the process thereof are similar to those described in the method for producing an intermediate antibody regioselectively modified.

### 2-3. Process (2)

The process (2) in the method for producing an antibody derivative regioselectively having a bioorthogonal functional group or bioorthogonal functional groups can be performed in the same manner as the process (2) in the method for producing an intermediate antibody regioselectively modified. Therefore, the definitions, examples, and preferable examples of terms such as mixture (first mixture) and reaction channel (first reaction channel) and the detail of embodiments such as conditions of the process thereof are similar to those described in the method for producing an intermediate antibody regioselectively modified.

### 2-4. Process (3)

The process (3) is performed by mixing the solution output from the first reaction channel with a solution comprising a cleavage agent introduced through a third inlet channel by a micromixer at the junction of the first reaction channel and the third inlet channel. By such mixing, a second mixture comprising the intermediate antibody and the cleavage agent is formed.

A cleavage agent having ability to cleave the cleavable site can be used as the cleavage agent. Examples of such a cleavage agent include reducing agents (e.g., tri(carboxyethyl)phosphine (TCEP), cysteine, dithiothreitol, reduced glutathione, and β-mercaptoethanol), acid substances (e.g., inorganic acid substances such as hydrochloric acid and sulfuric acid, and organic acid substances such as acetic acid and citric acid), basic substances (e.g., inorganic basic substances such as sodium hydroxide and potassium hydroxide, and organic basic substances such as hydroxylamine and triethylamine), oxidizers (e.g., sodium periodate and oxidized glutathione), and enzymes. The cleavage agent may be preferably a reducing agent, an acid substance, a basic substance, or an oxidizer and more preferably a reducing agent, an acid substance, or a basic substance.

The concentration of the cleavage agent in the solution is not limited as long as the concentration enables a sufficient reaction with the intermediate antibody and, for example, may be 0.05 to 30 mM. The concentration may be preferably 0.1 mM or more, more preferably 0.2 mM or more, further more preferably 0.3 mM or more, and particularly preferably 0.4 mM or more. The concentration may be 20 mM or less, 10 mM or less, 5 mM or less, 2 mM or less, or 1 mM or less. The concentration may be defined as an equivalent to the antibody. Thus, such a concentration may be, for example, 1 to 100 molar equivalents, preferably 1 to 50 molar equivalents (or 2 to 50 molar equivalents), more preferably 1 to 30 molar equivalents (or 3 to 30 molar equivalents), further more preferably 1 to 20 molar equivalents (or 4 to 20 molar equivalents), and particularly preferably 1 to 15 molar equivalents (or 5 to 15 molar equivalents) to the antibody.

The solution can be introduced into the third inlet channel in the same manner as the introduction of the solutions into the first and second inlet channels. The length, the representative diameter, the shape, and the material of the third inlet channel and the flow rate of the solution in the third inlet channel therefore may be the same as those of the first and second inlet channels.

In a specific embodiment, the flow rate of the solution in the first reaction channel may be 0.5 mL/min or more. The flow rate of the solution in the first reaction channel can be indirectly adjusted by adjusting factors such as the flow rates of the solutions in the upstream channels (e.g., the first inlet channel and the second inlet channel). The flow rate of the solution in the first reaction channel may be preferably 0.8 mL/min or more, more preferably 1.2 mL/min or more, further more preferably 1.5 mL/min or more, and particularly preferably 2.0 mL/min or more. Such a flow rate may be 40 mL/min or less, 30 mL/min or less, 20 mL/min or less, or 10 mL/min or less. More specifically, such a flow rate may be preferably 0.5 to 40 mL/min, more preferably 0.8 to 30 mL/min, further more preferably 1.2 to 20 mL/min, particularly preferably 1.5 to 10 mL/min, or 2.0 mL to 10 mL/min.

In a specific embodiment, the flow rate of the solution in the third inlet channel may be faster than any of (a) the flow rate of the solution in the first inlet channel and (b) the flow rate of the solution in the second inlet channel. With such a flow rate employed for the third inlet channel, the solutions collide strongly at the junction to produce more minute solution units, and therefore a homogenous solution can be formed more rapidly. The flow rate of the solution into the third inlet channel may be, for example, 1.2 times or more, 1.4 times or more, 1.6 times or more, 1.8 times or more, or 2.0 times or more of the flow rate (a) or (b). The flow rate of the solution in the third inlet channel may be 0.5 mL/min or more. The flow rate of the solution in the third inlet channel may be preferably 0.8 mL/min or more, more preferably 1.2 mL/min or more, further more preferably 1.5 mL/min or more, and particularly preferably 2.0 mL/min or more. Such a flow rate may be 40 mL/min or less, 30 mL/min or less, 20 mL/min or less, or 10 mL/min or less. More specifically, such a flow rate may be preferably 0.5 to 40 mL/min, more preferably 0.8 to 30 mL/min, further more preferably 1.2 to 20 mL/min, particularly preferably 1.5 to 10 mL/min, or 2.0 mL to 10 mL/min.

At the junction of the first reaction channel and the third inlet channel, a micromixer as described above is used. The definitions, examples, and preferable examples of the micromixer used in the present junction are similar to those of the micromixer described above used in the junction of the first inlet channel and the second inlet channel.

### 2-5. Process (4)

In the process (4), the second mixture obtained in the process (3) can be passed through a reaction channel to allow the intermediate antibody and the cleavage agent to react in the second reaction channel to form a solution comprising an antibody derivative regioselectively having a bioorthogonal functional group or bioorthogonal functional groups. When the second mixture is passed through the reaction channel, the cleavable site included in the intermediate antibody is cleaved by the cleavage agent. A solution comprising the antibody derivative regioselectively having a bioorthogonal functional group or bioorthogonal functional groups is thus formed.

The second reaction channel can be designed such that a desired residence time of the second mixture in the second reaction channel can be achieved in order to control the reaction time in the reaction between the antibody material and the cleavage agent. Such a residence time is not specifically limited, but may be, for example, less than 10 minutes, preferably less than 8 minutes, more preferably less than 5 minutes, and further more preferably less than 3 minutes. The residence time of the second mixture in the second reaction channel can be controlled, for example, by adjusting factors such as the flow rates of the solutions in the first, second, and third inlet channels and the length and the representative diameter of the second reaction channel.

The reaction in the second reaction channel can be performed under the above described mild condition that cannot induce denaturation or degradation of the antibody (e.g., cleavage of amide bonds). The reaction temperature in the second reaction channel can be readily controlled, in the same manner as the reaction temperature in the reaction channel described above.

The second reaction channel is not limited as long as it is constructed such that the residence time of the second mixture as described above can be achieved. For example, the length, the representative diameter, the shape, and the material of the second reaction channel may be similar to those of the reaction channel described above.

In a specific embodiment, the length and the representative diameter of the second reaction channel may be set such that the relation between the residence time in the second reaction channel and the residence time in the first reaction channel is adjusted. For example, the length and the representative diameter of the second reaction channel can be set such that the residence time of the second mixture in the second reaction channel is equal to or shorter than the residence time of the first mixture in the first reaction channel (preferably, ¾ or less or ½ or less). In this case, the length of the second reaction channel may be set to be equal to or smaller than the length of the first reaction channel (for example, a length of ¾ or less, ½ or less, or ¼ or less). The representative diameter of the second reaction channel may be set to be equal to or smaller than the representative diameter of the first reaction channel (for example, a representative diameter of ¾ or less, ½ or less, or ¼ or less).

According to the present invention, an antibody derivative regioselectively having a bioorthogonal functional group or bioorthogonal functional groups can be formed in a short time because the residence time of the first mixture in the first reaction channel can be set to be short and the residence time of the second mixture in the second reaction channel can be set to be equal to or shorter than the residence time of the first mixture in the first reaction channel.

In a specific embodiment, the time taken for formation of the antibody derivative regioselectively having a bioorthogonal functional group or bioorthogonal functional groups can be defined by the time required from arrival at the given micromixer to passage through the second reaction channel of the solution comprising an antibody material and the solution comprising the compound. Given that the mixing by the micromixer can be performed instantaneously, the time required for formation of the antibody derivative regioselectively having a bioorthogonal functional group or bioorthogonal functional groups can be mainly determined according to the residence time of the first mixture in the first reaction channel and the residence time of the second mixture in the second reaction channel. The residence time of the first mixture in the first reaction channel may be controlled to preferably within 3 minutes as described above in the formation of the intermediate antibody. The residence time of the second mixture in the second reaction channel may be controlled to within 1.5 minutes. For example, such control can be achieved by adjusting factors such as the flow rates of the solutions in the upstream channels (e.g., the first, second, and third inlet channels) and the length and the representative diameter of the second reaction channel. Preferably, the residence time of the mixture in the second reaction channel may be within 1 minute, within 50 seconds, within 40 seconds, within 30 seconds, or within 20 seconds. According to the present invention, therefore, the total time of the residence time of the first mixture in the first reaction channel and the residence time of the second mixture in the second reaction channel may be controlled to within 4.5 minutes. Preferably, the total time may be within 4 minutes, within 3.5 minutes, within 3 minutes, within 2.5 minutes, or within 2 minutes.

### 3. Method for Producing Antibody Derivative Regioselectively Having Functional Substance

### 3-1. Overview

The present invention provides a method for producing an antibody derivative regioselectively having a functional substance or functional substances. The method according to the present invention includes the following (I) to (III):
(I) forming (a) a solution comprising an intermediate antibody regioselectively having a bioorthogonal functional group or bioorthogonal functional groups by the method for producing an intermediate antibody regioselectively modified, or forming (b) a solution comprising an antibody derivative regioselectively having a bioorthogonal functional group or bioorthogonal functional groups by the method for producing an antibody derivative regioselectively having a bioorthogonal functional group or bioorthogonal functional groups;
(II) mixing the solution formed in (I) and a solution comprising a functional substance in a micromixer to form (a) a mixture comprising the intermediate antibody and the functional substance or (b) a mixture comprising the antibody derivative and the functional substance; and
(III) passing the mixture formed in (II) through a reaction channel to allow (a) the intermediate antibody and the functional substance or (b) the antibody derivative and the functional substance to react in the reaction channel to form a solution comprising an antibody derivative regioselectively having a functional substance or functional substances.

The processes (I) to (III) are continuously performed in a flow microreactor.

### 3-2. Process (I)

In the process (I) in the method for producing an antibody derivative regioselectively having a functional substance or functional substances, a solution comprising an intermediate antibody regioselectively having a bioorthogonal functional group or bioorthogonal functional groups can be formed by the method for producing an intermediate antibody regioselectively modified. Alternatively, a solution comprising an antibody derivative regioselectively having a bioorthogonal functional group or bioorthogonal functional groups can be formed by the method for producing an antibody derivative regioselectively having a bioorthogonal functional group or bioorthogonal functional groups.

### 3-3. Process (II)

The process (II) can be performed, for example, by mixing a solution output from the upstream reaction channel with a solution comprising a functional substance introduced through a fourth inlet channel by a micromixer at the junction of the upstream reaction channel and the fourth inlet channel. By such mixing, (a) a mixture comprising the intermediate antibody and the functional substance or (b) a mixture comprising the antibody derivative and the functional substance is formed.

The functional substance (also called payload) is not limited as long as the functional substance is a substance that imparts a given function to the antibody, and examples thereof include pharmaceuticals, labelling substances, and stabilizers. The functional substance may be a single functional substance or may be a substance in which two or more functional substances are coupled with each other.

For the medicine, a medicine for any given disease can be used. Examples of the disease include cancers (e.g., lung cancer, gastric cancer, colon cancer, pancreatic cancer, kidney cancer, liver cancer, thyroid cancer, prostate cancer, bladder cancer, ovarian cancer, uterine cancer, bone cancer, skin cancer, brain tumor, and melanoma), autoimmune diseases and inflammatory diseases (e.g., allergic disease, rheumatoid arthritis, and systemic lupus erythematosus), cranial nerve diseases (e.g., cerebral infarction, Alzheimer's disease, Parkinson's disease, and amyotrophic lateral sclerosis), infectious diseases (e.g., bacterial infection, and virus infection), genetic and rare diseases (e.g., hereditary spherocytosis, and non-dystrophic myotonia), eye diseases (e.g., age-related macular degeneration, diabetic retinopathy, and retinitis pigmentosa), diseases in the fields of bone and orthopedics (e.g., osteoarthritis), blood diseases (e.g., leukemia, and purpura), and other diseases (e.g., metabolic abnormality such as diabetes and hyperlipidemia, liver disease, kidney disease, lung disease, cardiovascular disease, and digestive system disease). The medicine may be a preventive or curative drug or a side effect reliever.

Preferably, the medicine is an anticancer drug. Examples of the anticancer drug include chemotherapeutic agents, toxins, radioisotopes, or substances containing them. Examples of the chemotherapeutic agents include DNA damaging agents, antimetabolites, enzyme inhibitors, DNA intercalating agents, DNA cleavage agents, topoisomerase inhibitors, DNA-binding inhibitors, tubulin-binding inhibitors, cytotoxic nucleoside, and platinum compounds.

Examples of the toxins include bacterial toxins (e.g., diphtheria toxin) and phytotoxin (e.g., ricin). Examples of the radioisotopes include radioisotopes of hydrogen (e.g., ³H), radioisotopes of carbon (e.g., ¹⁴C), radioisotopes of phosphor (e.g., ³²P), radioisotopes of sulfur (e.g., ³⁵S), radioisotopes of yttrium (e.g., ⁹⁰Y), radioisotopes of technetium (e.g., ^{99m}Tc), radioisotopes of indium (e.g., ¹¹¹In), radioisotopes of iodine (e.g., ¹²³I, ¹²⁵I, ¹²⁹I, and ¹³¹I), radioisotopes of samarium (e.g., 1⁵³Sm), radioisotopes of rhenium (e.g., ¹⁸⁶Re), radioisotopes of astatine (e.g., ²¹¹At), and radioisotopes of bismuth (e.g., ²¹²Bi). More specifically, examples of the medicine include auristatin (MMAE and MMAF), maytansine (DM1 and DM4), PBD (pyrrolobenzodiazepine), IGN, camptothecin analogues, calicheamicin, duocarmycin, eribulin, anthracycline, dmDNA31, and tubulysin.

The labeled substance is a substance that enables detection of a target (e.g., tissues, cells, and substances). Examples of the labeled substance include enzymes (e.g., peroxidase, alkaline phosphatase, luciferase, β-galactosidase), affinity substances (e.g., streptavidin, biotin, digoxigenin, and aptamer), fluorescent substances (e.g., fluorescein, fluorescein isothiocyanate, rhodamine, green fluorescent protein, and red fluorescent protein), luminous substances (e.g., luciferin, aequorin, acridinium ester, tris(2,2′-bipyridyl)ruthenium, and luminol), radioisotopes (e.g., those listed above), and substances containing them.

The stabilizer is a substance that enables stabilization of the antibody. Examples of the stabilizer include polymer compounds (e.g., polyethylene glycol (PEG)), diols, glycerin, nonionic surfactants, anionic surfactants, natural surfactants, saccharides, and polyols.

The functional substance may also be peptides, proteins (e.g., antibody), nucleic acids (e.g., DNA, RNA, and artificial nucleic acid), small compounds, chelators, glycans, lipids, polymer compounds, or metals (e.g., gold).

When the functional substance has a functional group that easily reacts with a bioorthogonal functional group, the functional group of the functional substance can react with the bioorthogonal functional group in the intermediate antibody or antibody derivative. The functional group that easily reacts with a bioorthogonal functional group may vary according to specific kinds of bioorthogonal functional groups. Those skilled in the art could select an appropriate functional group as appropriate as the functional group that easily reacts with a bioorthogonal functional group (e.g., Boutureira et al., Chem. Rev., 2015, 115, 2174-2195, which is incorporated herein by reference in its entirety). Examples of the functional group easily reacting with the bioorthogonal functional group include, but not limited to, an alkyne residue when the bioorthogonal functional group is an azide residue, a maleimide residue and a disulfide residue when the bioorthogonal functional group is a thiol residue, a hydrazine residue when the bioorthogonal functional group is an aldehyde residue or a ketone residue, an azide residue when the bioorthogonal functional group is a norbornene residue, and an alkyne residue when the bioorthogonal functional group is a tetrazine residue. As a matter of course, for the above combination of the bioorthogonal functional group and the functional group easily reacting therewith, the components of the combination can be replaced each other. Therefore, when the components of the first example in the above combination are replaced each other, a combination of the alkyne residue as the bioorthogonal functional group and the azide residue as the functional group easily reacting with the bioorthogonal functional group can be used.

When the functional substance does not have a functional group that easily reacts with the bioorthogonal functional group in the intermediate antibody or antibody derivative, the drug may be derivatized to have such a functional group. The derivatization is technically common in the art (e.g., WO2004/010957, specification of United States Patent Application Publication No. 2006/0074008, and specification of United States Patent Application Publication No. 2005/0238649, which are incorporated herein by reference in their entireties). For example, the derivatization may be performed using any cross-linker. Alternatively, the derivatization may be performed by a specific linker having a desired functional group. For example, such a linker is the one that can separate the drug and the antibody by cleaving the linker in an appropriate environment (e.g., inside the cell or outside the cell). Examples of such a linker include peptidyl linkers decomposed in specific proteases [e.g., intracellular proteases (e.g., protease present in a lysosome or an endosome), extracellular proteases (e.g., secreted protease)] (e.g., U.S. Pat. No. 6,214,345; Dubowchik et al., Pharm. Therapeutics 83: 67-123 (1999), which are incorporated herein by reference in their entireties), and linkers that can be cleaved at local acidic sites present in living bodies (e.g., U.S. Pat. Nos. 5,622,929, 5,122,368; and 5,824,805, which are incorporated herein by reference in their entireties). The linker may be self-immolative (e.g., WO02/083180, WO04/043493, and WO05/112919, which are incorporated herein by reference in their entireties). In the present invention, the derivatized functional substance is also simply referred to as “functional substance”.

In a specific embodiment, the functional substance may have a maleimide group and/or a disulfide group or may be derivatized to have a maleimide group and/or a disulfide group.

The concentration of the functional substance in the solution is not limited as long as the concentration enables a sufficient reaction with the intermediate antibody or the antibody derivative as described above and, for example, may be 0.05 to 30 mM. The concentration may be preferably 0.1 mM or more, more preferably 0.2 mM or more, further more preferably 0.3 mM or more, or particularly preferably 0.4 mM or more. The concentration may be 20 mM or less, 10 mM or less, 5 mM or less, 2 mM or less, or 1 mM or less. The concentration may be defined as an equivalent to the antibody. Thus, such a concentration may be, for example, 1 to 100 molar equivalents, preferably 1 to 50 molar equivalents (or 2 to 50 molar equivalents), more preferably 1 to 30 molar equivalents (or 3 to 30 molar equivalents), further more preferably 1 to 20 molar equivalents (or 4 to 20 molar equivalents), and particularly preferably 1 to 15 molar equivalents (or 5 to 15 molar equivalents) to the antibody.

The solution can be introduced into the fourth inlet channel in the same manner as the introduction of the solutions into the first and second inlet channels. For example, the length, the representative diameter, the shape, and the material of the fourth inlet channel and the flow rate of the solution in the fourth inlet channel may be the same as those of the first and second inlet channels.

In a specific embodiment, the flow rate of the solution in the upstream reaction channel may be 1.0 mL/min or more. The flow rate of the solution in the upstream reaction channel can be indirectly adjusted by adjusting factors such as the flow rates of the solutions in the upstream inlet channels (e.g., the first, second, and third inlet channels). The flow rate of the solution in the upstream reaction channel may be preferably 1.2 mL/min or more, more preferably 1.5 mL/min or more, further more preferably 1.8 mL/min or more, and particularly preferably 2.0 mL/min or more. Such a flow rate may be 40 mL/min or less, 30 mL/min or less, 20 mL/min or less, or 10 mL/min or less. More specifically, such a flow rate may be preferably 1.2 mL to 40 mL/min, more preferably 1.5 to 30 mL/min, further more preferably 1.8 to 20 mL/min, and particularly preferably 2.0 mL to 10 mL/min.

In a specific embodiment, the flow rate of the solution in the fourth inlet channel may be faster than any of (a) the flow rate of the solution in the first inlet channel, (b) the flow rate of the solution in the second inlet channel, and (c) the flow rate of the solution in the third inlet channel. With such a flow rate employed for the fourth inlet channel, the solutions collide strongly at the junction to produce more minute solution units, and therefore a homogenous solution can be formed more rapidly. The flow rate of the solution into the fourth inlet channel may be, for example, 1.2 times or more, 1.4 times or more, 1.6 times or more, 1.8 times or more, or 2.0 times or more of the flow rates (a) to (c). The flow rate of the solution in the fourth inlet channel may be 1.0 mL/min or more. The flow rate of the solution in the fourth inlet channel may be preferably 1.2 mL/min or more, more preferably 1.5 mL/min or more, further more preferably 1.8 mL/min or more, and particularly preferably 2.0 mL/min or more. Such a flow rate may be 40 mL/min or less, 30 mL/min or less, 20 mL/min or less, or 10 mL/min or less. More specifically, such a flow rate may be preferably 1.2 mL to 40 mL/min, more preferably 1.5 to 30 mL/min, further more preferably 1.8 to 20 mL/min, and particularly preferably 2.0 mL to 10 mL/min.

At the junction of the upstream reaction channel and the fourth inlet channel, a micromixer as described above is used. The definition, examples, and preferable examples of the micromixer used in the present junction are similar to those of the micromixer described above used in the junction of the first inlet channel and the second inlet channel.

### 3-4. Process (III)

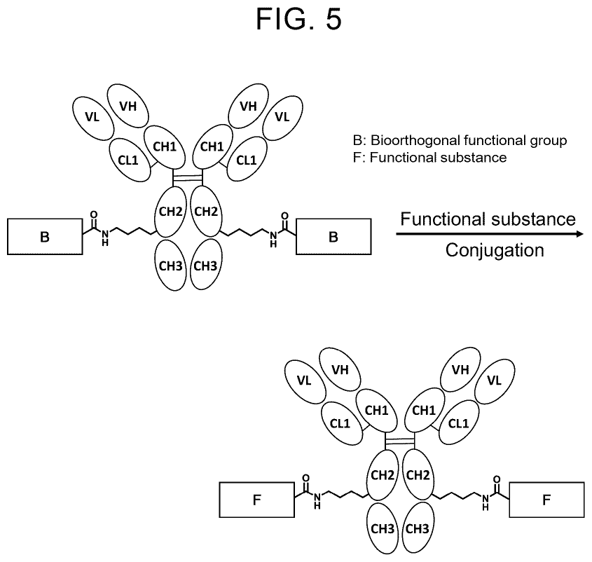

In the process (III), the mixture obtained in (II) can be passed through a reaction channel to allow (a) the intermediate antibody and the functional substance or (b) the antibody derivative and the functional substance to react in the reaction channel to form a solution comprising an antibody derivative regioselectively having a functional substance or functional substances. When the mixture is passed through the reaction channel, the bioorthogonal functional group included in the intermediate antibody or the antibody derivative reacts with the functional substance. A solution comprising an antibody derivative regioselectively having a functional substance or functional substances is thus formed (FIG. 5).

The reaction channel can be designed such that a desired residence time of the mixture in the reaction channel can be achieved in order to control the reaction time in the reaction between the antibody material and the functional substance. Such a residence time is not specifically limited, but may be, for example, less than 10 minutes, preferably less than 8 minutes, more preferably less than 5 minutes, and further more preferably less than 3 minutes. The residence time of the mixture in the reaction channel can be controlled, for example, by adjusting factors such as the flow rates of the solutions in the first, second, third, and fourth inlet channels and the length and the representative diameter of the reaction channel.

The reaction in the reaction channel can be performed under the above described mild condition that cannot induce denaturation or degradation of the antibody (e.g., cleavage of amide bonds). The reaction temperature in the reaction channel can be readily controlled, in the same manner as the reaction temperature in the other reaction channels described above.

The reaction channel is not limited as long as it is constructed such that the residence time of the mixture as described above can be achieved. For example, the length, the representative diameter, the shape, and the material of the reaction channel may be similar to those of the other reaction channels described above.

In a specific embodiment, the length and the representative diameter of the reaction channel may be set such that the relation between the residence time in the reaction channel and the residence time in the other reaction channels (e.g., the first and/or second reaction channel) is adjusted. For example, the length and the representative diameter of the reaction channel may be set such that the residence time of the mixture in the reaction channel is equal to or shorter than the residence time of the first mixture in the first reaction channel (preferably, ¾ or less or ½ or less). In this case, the length of the reaction channel may be set to be equal to or smaller than the length of the first reaction channel (preferably, a length of ¾ or smaller, ½ or smaller, or ¼ or smaller). The representative diameter of the reaction channel may be set to be equal to or smaller than the representative diameter of the first reaction channel (preferably, a representative diameter of ¾ or less, ½ or less, or ¼ or less).

According to the present invention, an antibody derivative regioselectively having a functional substance or functional substances can be formed in a short time because the residence time of the first mixture in the first reaction channel can be set to be short, the residence time of the second mixture in the second reaction channel can be set to be equal to or shorter than the residence time of the first mixture in the first reaction channel, and the residence time of the mixture in the reaction channel in the process (III) can also be set to be equal to or shorter than the residence time of the first mixture.

In a specific embodiment, the time taken for formation of the antibody derivative regioselectively having a functional substance or functional substances can be defined by the time required from arrival at the given micromixer to passage through the reaction channel in the process (III) of the solution comprising an antibody material and the solution comprising the compound. Given that the mixing by the micromixer can be performed instantaneously, the time required for formation of the antibody derivative regioselectively having a functional substance can be mainly determined according to the total residence time in all of the reaction channels used. The residence time of the first mixture in the first reaction channel may be controlled to preferably within 3 minutes as described above in the formation of the intermediate antibody. The residence time of the second mixture in the second reaction channel may be controlled to preferably within 1.5 minutes as described above in the formation of the antibody derivative. Furthermore, the residence time in the reaction channel in the process (III) may be controlled to within 1.5 minutes. For example, such control can be achieved by adjusting factors such as the flow rates of the solutions in the first, second, third, and fourth inlet channels and the length and the representative diameter of the reaction channel in the process (III). Preferably, the residence time of the mixture in the reaction channel in the process (III) may be within 1 minute, within 50 seconds, within 40 seconds, within 30 seconds, or within 20 seconds. According to the present invention, therefore, the total residence time in all of the reaction channels may be controlled to within 6 minutes. Preferably, the total residence time may be within 5.5 minutes, within 5 minutes, within 4.5 minutes, within 4 minutes, within 3.5 minutes, or within 3 minutes.

### 4. Antibody Obtained by Methods According to the Present Invention

The methods according to the present invention can produce a satisfactory intermediate antibody and antibody derivative in a range of 1.5 to 2.5 per immunoglobulin unit comprising two light chains and two heavy chains for the modification ratio of the compound to the intermediate antibody regioselectively modified (modification by the compound/antibody), the modification ratio of the bioorthogonal functional group to the intermediate antibody or the antibody derivative regioselectively having a bioorthogonal functional group or bioorthogonal functional groups (bioorthogonal functional group/antibody), and the modification ratio of the functional substance to the antibody derivative regioselectively having the functional substance (functional substance/antibody). Such a modification ratio may be in a range of preferably 1.6 to 2.4, more preferably 1.7 to 2.3, further more preferably 1.8 to 2.2, and particularly preferably 1.9 to 2.1 (typically 2.0). Such a modification ratio can be determined according to WO 2019/240287 (WO 2019/240287A1, which are incorporated herein by reference in their entireties) using ESI-TOFMS analysis and a DAR calculator (software from Agilent Technologies, Inc.).

Furthermore, the methods according to the present invention can reduce production of undesired byproducts (aggregates and fragmented antibody decomposed products) in the production of an intermediate antibody regioselectively modified, an intermediate antibody or an antibody derivative regioselectively having a bioorthogonal functional group or bioorthogonal functional groups, and an antibody derivative regioselectively having a functional substance or functional substances. The intermediate antibody and the antibody derivative produced by the methods according to the present invention therefore can be defined by their purity. The purity of the intermediate antibody and the antibody derivative can be evaluated by the monomer ratio of the intermediate antibody and the antibody derivative. As used herein the monomer ratio refers to the ratio of non-aggregated and non-decomposed antibody to the entire antibody (in other words, the antibody other than the byproducts). The monomer ratio of the intermediate antibody and the antibody derivative may be, for example, 98% or more, preferably 98.5% or more, more preferably 99% or more, and further more preferably 99.5% or more. In the present invention, the monomer ratio of the intermediate antibody and the antibody derivative can be measured by size exclusion chromatography (SEC) according to the previous report (Chemistry Select 2020, 5, 8435-8439, which is incorporated herein by reference in its entirety).

The intermediate antibody or the antibody derivative formed by the reaction in the final reaction channel can be collected and purified as appropriate. For example, the intermediate antibody or the antibody derivative can be collected by a container (e.g., fraction collector) disposed at the exit of the reaction channel. For example, the collected intermediate antibody or antibody derivative can be purified by any method such as chromatography (e.g., gel filtration chromatography, ion exchange chromatography, reversed-phase column chromatography, high performance liquid chromatography, and affinity chromatography). The purification may be performed continuously in the FMR. For example, in such a case, a purification channel for the intermediate antibody or the antibody derivative may be further disposed further downstream of the final reaction channel.

Other features of the invention will become apparent in the course of the following descriptions of exemplary embodiments which are given for illustration of the invention and are not intended to be limiting thereof.

### EXAMPLES

It should be noted that the reagent for regioselectively modifying an antibody (compound) as described above is referred to as alternative expressions such as affinity peptide reagent, affinity peptide, and peptide reagent.

### Overview of Flow Microreactor (FMR)

The overview of the configuration of the FMR used in Examples below is described below.

In Examples, channels, micromixers, and reaction tubes having a circular shape in channel section were used. In the following, the term “inner diameter” is therefore used as a representative diameter.

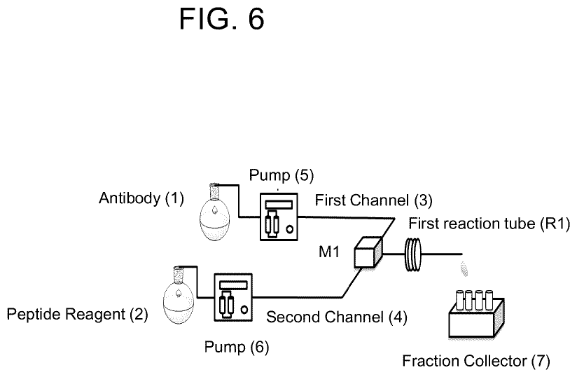

The overview of the FMR for mixing two solutions is as described below and illustrated in FIG. 6.

### 1) Reservoir

First reservoir (1) containing an antibody solution
Second reservoir (2) containing an affinity peptide reagent solution
   2) Channel from Reservoir to Micromixer
First inlet channel (3) for introducing the antibody solution to connect the first reservoir (1) to a first micromixer (M1)
Second inlet channel (4) for introducing the affinity peptide reagent solution to connect the second reservoir (2) to the first micromixer (M1)

### 3) Pump

First pump (5) provided for the first channel (3)
Second pump (6) provided for the second channel (4)

### 4) Micromixer

First micromixer (M1) for forming a mixture of the antibody solution and the affinity peptide reagent solution

A T-shaped micromixer for merging the first channel and the second channel facing each other was used as the first micromixer.

The inner diameter and the shape of the channel in the first micromixer (M1) are listed in Table 1. The inner diameter of the channel indicates the channel width of the mixing unit of two kinds of solutions.

### 5) Reaction Tube

First reaction tube (R1) for allowing an antibody and an affinity peptide reagent in the mixture of the antibody solution and the affinity peptide reagent solution to react

### 6) Fraction Collector

Fraction collector (7) for collecting a first reaction solution from the first reaction tube

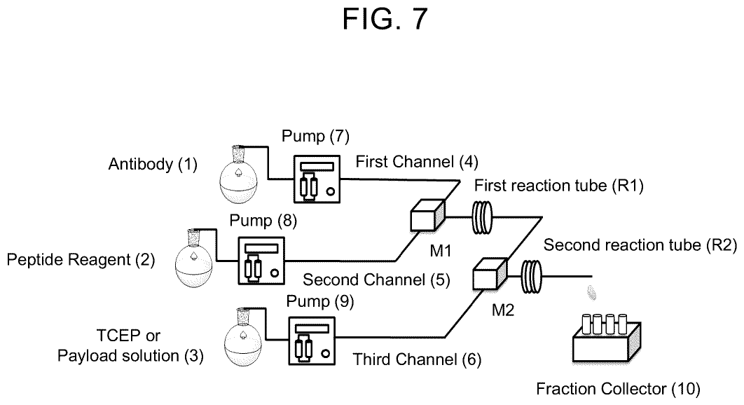

The overview of the FMR for mixing three solutions is as described below and illustrated in FIG. 7.

### 1) Reservoir

First reservoir (1) containing an antibody solution
Second reservoir (2) containing an affinity peptide reagent solution
Third reservoir (3) containing a reducing agent solution or a payload solution
   2) Channel from Reservoir to Micromixer
First inlet channel (4) for introducing the antibody solution to connect the first reservoir (1) to a first micromixer (M1)
Second inlet channel (5) for introducing the affinity peptide reagent solution to connect the second reservoir (2) to the first micromixer (M1)
Third inlet channel (6) for introducing the reducing agent solution or the payload solution to connect the third reservoir (3) to a second micromixer (M2)

### 3) Pump

First pump (7) provided for the first channel (3)
Second pump (8) provided for the second channel (4)
Third pump (9) provided for the third channel (6)

### 4) Micromixer

First micromixer (M1) for forming a mixture of the antibody solution and the affinity peptide reagent solution
Second micromixer (M2) for forming a second mixture of a first reaction solution and the reducing agent solution or the payload solution

T-shaped micromixers for merging the first channel and the second channel facing each other were used as the first and second micromixers.

The inner diameters and the shapes of the channels in the first micromixer (M1) and the second micromixer (M2) are listed in Table 1. The inner diameter of the channel indicates the channel width of the mixing unit of two kinds of solutions.

### 5) Reaction Tube

First reaction tube (R1) for allowing an antibody and an affinity peptide reagent in the mixture of the antibody solution and the affinity peptide reagent solution to react
Second reaction tube (R2) for allowing a peptide-added antibody and a reducing agent or a payload in the mixture of the first reaction solution and the payload solution to react

### 6) Fraction Collector

Fraction collector (10) for collecting a second reaction solution from the second reaction tube

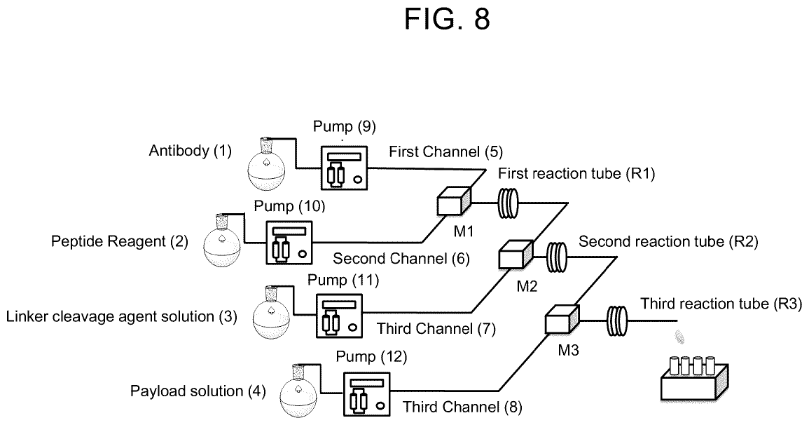

The overview of the FMR for mixing four solutions is as described below and illustrated in FIG. 8.

### 1) Reservoir

First reservoir (1) containing an antibody solution
Second reservoir (2) containing an affinity peptide reagent solution
Third reservoir (3) containing a linker cleavage agent solution
Fourth reservoir (4) containing a payload solution
   2) Channel from Reservoir to Micromixer
First inlet channel (5) for introducing the antibody solution to connect the first reservoir (1) to a first micromixer (M1)
Second inlet channel (6) for introducing the affinity peptide reagent solution to connect the second reservoir (2) to the first micromixer (M1)
Third inlet channel (7) for introducing the linker cleavage agent solution to connect the third reservoir (3) to a second micromixer (M2)
Fourth inlet channel (8) for introducing the payload solution to connect the fourth reservoir (4) to a third micromixer (M3)

### 3) Pump

First pump (9) provided for the first channel (5)
Second pump (10) provided for the second channel (6)
Third pump (11) provided for the third channel (7)
Fourth pump (12) provided for the fourth channel (8)

### 4) Micromixer

First micromixer (M1) for forming a mixture of the antibody solution and the affinity peptide reagent solution
Second micromixer (M2) for forming a second mixture of a first reaction solution and the reducing agent solution
Third micromixer (M3) for forming a third mixture of a second reaction solution and the payload solution

T-shaped micromixers for merging the first channel, the second channel, and the third channel facing each other were used as the first, second, and third micromixers.

The inner diameters and the shapes of the channels in the first micromixer (M1), the second micromixer (M2), and the third micromixer (M3) are listed in Table 1. The inner diameter of the channel indicates the channel width of the mixing unit of two kinds of solutions.

### 5) Reaction Tube

First reaction tube (R1) for allowing an antibody and an affinity peptide reagent in the mixture of the antibody solution and the affinity peptide reagent solution to react
Second reaction tube (R2) for allowing linker cleavage to proceed in the mixture of the first reaction solution and the linker cleavage agent solution
Second reaction tube (R3) for allowing a linker antibody and a payload in the mixture of the second reaction solution and the payload solution to react

### 6) Fraction Collector

Fraction collector (13) for collecting a third reaction solution from the third reaction tube

### Temperature Adjustment

For temperature adjustment, the first reaction tube (R1), the second reaction tube (R2), and the third reaction tube (R3) were designed such that a coil residence tube for pre-temperature adjustment and a water bath for temperature adjustment can be used, if necessary.

### Specifications of Channels, Micromixers, and Reaction Tubes

The lengths of the first, second, third, and fourth inlet channels are 1.0 m. The inner diameter of the first inlet channel is 1.0 mm, the inner diameter of the second inlet channel is 1.0 mm, the inner diameter of the third inlet channel is 1.0 mm, and the inner diameter of the fourth inlet channel is 1.0 mm.

The inner diameters and the shapes of the channels in the first micromixer (M1), the second micromixer (M2), and the third micromixer (M3) are listed in Table 1.

The inner diameters and the lengths of the first reaction tube (R1), the second reaction tube (R2), and the third reaction tube (R3) are listed in Table 2.

TABLE 1
Inner diameter and shape of channel in micromixer

| Micromixer | Inner diameter Y | Shape |
|---|---|---|
| First (M1) | 0.25 mm | T-shaped |
| Second (M2) | 0.25 mm | T-shaped |
| Third (M3) | 0.25 mm | T-shaped |

TABLE 2
Inner diameter and length of channel in reaction tube

| Reaction tube | Inner diameter | Length |
|---|---|---|
| First (R1) | 1.0 mm | 0.33, 0.66, 2.0, 4.0, |
| | | 8.0 and 16.0 m |
| Second (R2) | 1.0 mm | 4.0 m |
| Third (R3) | 1.0 mm | 4.0 m |

In Examples below, the following reaction was performed using the thus constructed FMR apparatus. When the mixing temperature was changed, the micromixer was immersed in a water bath. The mixing temperature was set to 25° C. unless otherwise specified.

### (A) Regioselective Modification Reaction of Antibody by Affinity Peptide Reagent

An antibody-affinity peptide modified compound was synthesized within 3 minutes of the residence time, using a peptide reagent having affinity to an antibody. More specifically, the antibody-affinity peptide conjugate was prepared by feeding each of an antibody solution and an affinity peptide solution by a pump, mixing the solutions in the first micromixer, passing the mixture through the first reaction tube, and collecting the solution by the fraction collector.

### (B) Integration of Regioselective Modification Reaction of Antibody by Affinity Peptide Reagent and Subsequent Reduction Reaction

A reduced antibody in which a modification group was regioselectively introduced (an antibody in which a heavy chain and a light chain are dissociated by a reducing agent TCEP. An antibody in which the structure of the antibody was retained in a noncovalent binding manner even after reduction, and consequently affinity to an antigen is kept) was synthesized within 1.7 minutes of the residence time, using a peptide reagent having affinity to an antibody. More specifically, the reduced antibody in which a modification group was regioselectively introduced was prepared by feeding each of an antibody solution and an affinity peptide solution by a pump, mixing the solutions in the first micromixer, passing the mixture through the first reaction tube, feeding and mixing a reducing agent solution with the mixture in the second micromixer, passing the mixture through the second reaction tube, and collecting the solution by the fraction collector.

### (C) Synthesis of Regioselective ADC

A regioselective ADC was synthesized within 4.5 minutes of the residence time, using a peptide reagent having affinity to an antibody. More specifically, the regioselective ADC was prepared by feeding each of an antibody solution and an affinity peptide solution by a pump, mixing the solutions in the first micromixer, passing the mixture through the first reaction tube, feeding and mixing a payload solution with the mixture in the second micromixer, passing the mixture through the second reaction tube, and collecting the solution by the fraction collector.

### (D) Synthesis of Regioselective ADC

A regioselective ADC was synthesized within 6 minutes of the residence time, using a peptide reagent having affinity to an antibody. More specifically, the regioselective ADC was prepared by feeding each of an antibody solution and an affinity peptide solution by a pump, mixing the solutions in the first micromixer, passing the mixture through the first reaction tube, feeding and mixing a linker cleavage agent solution with the mixture in the second micromixer, passing the mixture through the second reaction tube, feeding and mixing a payload solution with the mixture in the third micromixer, passing the mixture through the third reaction tube, and collecting the solution by the fraction collector.

### Example 1: Synthesis of Trastuzumab-Affinity Peptide Conjugate

### (1-1) Synthesis of Trastuzumab-Affinity Peptide Conjugate Using Affinity Peptide Reagent (1)

### (1-1-1) Conjugation of Affinity Peptide Reagent (1) and Trastuzumab

The influence of the conjugation reaction time on the peptide-to-antibody ratio (PAR) of the trastuzumab-affinity peptide conjugate was examined.

The first reservoir (1) of the FMR (FIG. 6) was filled with an anti-HER2 IgG antibody trastuzumab (Chugai Pharmaceutical Co., Ltd.) 10 mg/mL 50 mM sodium acetate buffer solution (pH=5.5). The second reservoir (2) was filled with a 0.667 mM solution of an affinity peptide 1 (compound 1) in the previous report (WO 2019/240287 (WO 2019/240287A1, which are incorporated herein by reference in their entireties), Example 84) with a conjugation buffer solution (sodium acetate buffer:DMF=9:1, pH 5.5). The antibody solution introduced at a flow rate of 1.0 mL/min and the affinity peptide solution introduced at a flow rate of 1.0 mL/min were mixed in the first micromixer (M1) and subjected to a conjugation reaction in the first reaction tube (R1). The conjugation reaction time is listed in Table 3. The solution after the conjugation reaction in the first reaction tube (R1) was collected by the fraction collector to which an excessive amount of L-lysine was added in advance. L-lysine is used for stopping the conjugation reaction after the collection by the fraction collector in order to accurately evaluate the PAR of the trastuzumab-affinity peptide conjugate formed by the conjugation reaction while the solution is passing through the first reaction tube (R1). Subsequently, the PAR of the ADC included in the collected fraction was measured by ESI-TOFMS analysis. The result is listed in Table 3.

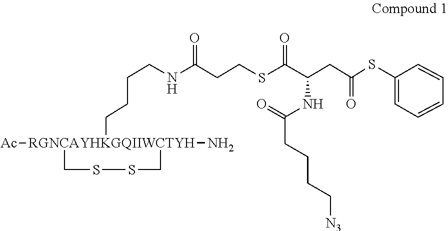

(Amino acid sequence of peptide portion: SEQ ID NO: 12)

TABLE 3
Relation between conjugation reaction
conditions and PAR in synthesis of ADC

| Condition | Residence time | | R1 (m) | PAR |
|---|---|---|---|---|
| 1 | 6 | min | 16 | 2.0 |
| 2 | 3 | min | 8 | 2.0 |
| 3 | 1.5 | min | 4 | 1.2 |
| 4 | 0.75 | min | 2 | 0.3 |
| 5 | 0.25 | min | 0.66 | 0.1 |
| 6 | 0.166 | min | 0.44 | 0.0 |
| 7 | 0.125 | min | 0.33 | 0.0 |

R1 indicates the length of the channel in the first reaction tube.

The residence time corresponds to the time during which the mixture of the antibody and the affinity peptide reagent stays in the first reaction tube (R1).

### (1-1-2) PAR Analysis of Trastuzumab-Affinity Peptide Conjugate

ESI-TOFMS analysis of the trastuzumab-affinity peptide conjugate obtained under condition 1 in Table 3 in (1-1-1) was performed according to the previous report (WO 2019/240287 (WO 2019/240287A1), which are incorporated herein by reference in their entireties). For the raw material trastuzumab, a peak was observed at 148,222. For a reaction product, a peak was observed at 153,026 with two modifying reagents (1) introduced.

The determination of the peptide/antibody binding ratio using DAR calculator (software from Agilent Technologies, Inc.) was subsequently performed and the result is listed in Table 3. The average peptide/antibody binding ratio calculated from the PAR peak in Table 3 and % Area was 2.0.

### (1-1-3) Determination of Heavy Chain Selectivity of Trastuzumab-Affinity Peptide Conjugate

ESI-TOFMS analysis under reduction conditions of the trastuzumab-affinity peptide conjugate obtained under condition 1 in Table 3 in (1-1-1) was performed according to the previous report (WO 2019/240287 (WO 2019/240287A1), which are incorporated herein by reference in their entireties). For the raw material trastuzumab, heavy chain peaks were observed at 50,594 and 50,755 and a light chain peak was observed at 23,439, and for a reaction product, peaks were observed at 52,909 and 53,070 with a linker introduced to the heavy chain, and a light chain peak was observed at 23,439, the same as that of the raw material.

### (1-1-4) Monomer Ratio Analysis of Trastuzumab-Affinity Peptide Conjugate

The trastuzumab-affinity peptide conjugate obtained under condition 1 in Table 3 in (1-1-1) was subjected to size exclusion chromatography (SEC) analysis according to the previous report (Chemistry Select 2020, 5, 8435-8439, which is incorporated herein by reference in its entirety). More specifically, the analysis of the ratio of monomer (a unit comprising two light chains and two heavy chains of IgG) in the antibody-drug conjugate by SEC is as follows. Advance Bio SEC 300 Å (manufactured by Agilent Technologies, Inc.) was used as a column, and 100 mM NaHPO₄/NaH₂PO₄, 250 mM NaCl, and 10% v/v isopropanol (2-propanol), pH 6.8 were used as eluents. An ADC sample (1 mg/mL) dissolved in a buffer was injected in the amount of 40 μL to HPLC and eluted for 11 minutes.

As a result, although a conjugation reaction was performed in an FMR, the monomer ratio exceeded 99%.

This indicated that in the affinity modification reaction by the affinity peptide reagent using the FMR, the reaction proceeded in a heavy chain selective manner in a short time to yield a conjugate with PAR=2.0.

### (1-2) Synthesis of Trastuzumab-Affinity Peptide Conjugate Using Affinity Peptide Reagent (2)

### (1-2-1) Conjugation of Affinity Peptide Reagent (2) and Trastuzumab

For trastuzumab, a conjugation reaction was attempted using an affinity peptide reagent (2) in the previous report (WO 2019/240287 (WO 2019/240287A1, which are incorporated herein by reference in their entireties), Example 81). Condition 2 in Table 3 in (1-1-1) was selected as the reaction condition.

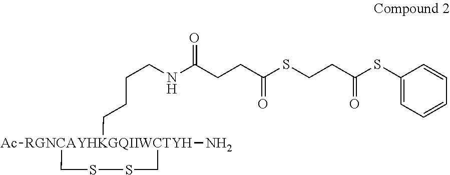

(Amino acid sequence of peptide portion: SEQ ID NO: 12)

### (1-2-2) PAR Analysis of Trastuzumab-Affinity Peptide Conjugate

ESI-TOFMS analysis of the trastuzumab-affinity peptide conjugate obtained in (1-2-1) was performed according to the condition in Example 1-1-2. For the raw material trastuzumab, a peak was observed at 148,222. For a reaction product, a peak was observed at 152,896 with two modifying reagents (2) introduced.

The determination of the peptide/antibody binding ratio using DAR calculator (software from Agilent Technologies, Inc.) was subsequently performed and the result is listed in Table 3. The average peptide/antibody binding ratio calculated from the PAR peak in Table 3 and % Area was 2.0.

### (1-2-3) Determination of Heavy Chain Selectivity of Trastuzumab-Affinity Peptide Conjugate

ESI-TOFMS analysis under reduction conditions of the trastuzumab-affinity peptide conjugate obtained in (1-2-1) was performed according to the condition in Example 1-1-3.

For the raw material trastuzumab, heavy chain peaks were observed at 50,594 and 50,755 and a light chain peak was observed at 23,439, and for a reaction product, peaks were observed at 52,855 and 53,017 with a linker introduced to the heavy chain, and a light chain peak was observed at 23,439, the same as that of the raw material.

### (1-2-4) Monomer Ratio Analysis of Trastuzumab-Affinity Peptide Conjugate

The trastuzumab-affinity peptide conjugate obtained in (1-2-1) was subjected to size exclusion chromatography (SEC) analysis according to Example 1-1-4.

As a result, although a conjugation reaction was performed in an FMR, the monomer ratio exceeded 99%.

### (1-3) Synthesis of Trastuzumab-Affinity Peptide Conjugate Using Affinity Peptide Reagent (3)

### (1-3-1) Conjugation of Affinity Peptide Reagent (3) and Trastuzumab

For trastuzumab, a conjugation reaction was attempted using an affinity peptide reagent (3) in the previous report (Angew. Chem. Int. Ed., 2019, 58, 5592-5597, which is incorporated herein by reference in its entirety). Condition 6 in Table 3 in (1-1-1) was selected as the reaction condition.

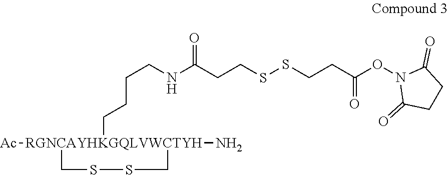

(Amino acid sequence of peptide portion: SEQ ID NO: 13)

### (1-3-2) DAR Analysis of Trastuzumab-Affinity Peptide Conjugate

ESI-TOFMS analysis of the trastuzumab-affinity peptide conjugate obtained in (1-3-1) was performed according to the condition in Example 1-1-2. For the raw material trastuzumab, a peak was observed at 148,222. For a reaction product, a peak was observed at 152,721 with two modifying reagents (3) introduced.

The determination of the peptide/antibody binding ratio using DAR calculator (software from Agilent Technologies, Inc.) was subsequently performed. The average peptide/antibody binding ratio calculated from the obtained PAR peak and % Area was 2.0, and a trastuzumab-affinity peptide conjugate with PAR=2.0 was obtained in a residence time as short as 10 seconds when compound 3 was used as the affinity substance.

### (1-3-3) Determination of Heavy Chain Selectivity of Trastuzumab-Affinity Peptide Conjugate

ESI-TOFMS analysis under reduction conditions of the trastuzumab-affinity peptide conjugate obtained in (1-3-1) was performed according to the condition in Example 1-1-3. For the raw material trastuzumab, heavy chain peaks were observed at 50,594 and 50,755 and a light chain peak was observed at 23,439, and for a reaction product, peaks were observed at 50,683 and 50,845 with a linker introduced to the heavy chain, and a light chain peak was observed at 23,439, the same as that of the raw material.

### (1-3-4) Monomer Ratio Analysis of Trastuzumab-Affinity Peptide Conjugate

The trastuzumab-affinity peptide conjugate obtained in (1-3-1) was subjected to size exclusion chromatography (SEC) analysis according to Example 1-1-4.

As a result, although a conjugation reaction was performed in an FMR, the monomer ratio exceeded 99%.

### (1-4) Synthesis of Trastuzumab-Affinity Peptide Conjugate Using Affinity Peptide Reagent (4)

### (1-4-1) Conjugation of Affinity Peptide Reagent (4) and Trastuzumab

For trastuzumab, a conjugation reaction was attempted using an affinity peptide reagent (4) in the previous report (Angew. Chem. Int. Ed., 2019, 58, 5592-5597, which is incorporated herein by reference in its entirety). Condition 6 in Table 3 in (1-1-1) was selected as the reaction condition.

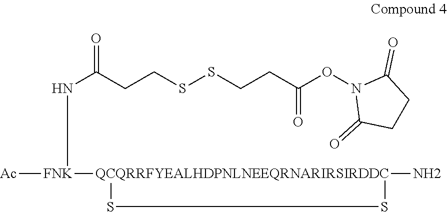

(Amino acid sequence of peptide portion: SEQ ID NO: 14)

### (1-4-2) PAR Analysis of Trastuzumab-Affinity Peptide Conjugate

ESI-TOFMS analysis of the trastuzumab-affinity peptide conjugate obtained in (1-4-1) was performed according to the condition in Example 1-1-2. For the raw material trastuzumab, a peak was observed at 148,222. For a reaction product, a peak was observed at 157,124 with two modifying reagents (4) introduced.

The determination of the peptide/antibody binding ratio using DAR calculator (software from Agilent Technologies, Inc.) was subsequently performed. The average peptide/antibody binding ratio calculated from the obtained PAR peak and % Area was 2.0, and a trastuzumab-affinity peptide conjugate with PAR=2.0 was obtained in a residence time as short as 10 seconds when compound 3 was used as the affinity substance.

### (1-4-3) Determination of Heavy Chain Selectivity of Trastuzumab-Affinity Peptide Conjugate

ESI-TOFMS analysis under reduction conditions of the trastuzumab-affinity peptide conjugate obtained in (1-4-1) was performed according to the condition in Example 1-1-3.

For the raw material trastuzumab, heavy chain peaks were observed at 50,594 and 50,755 and a light chain peak was observed at 23,439, and for a reaction product, peaks were observed at 50,683 and 50,845 with a linker introduced to the heavy chain, and a light chain peak was observed at 23,439, the same as that of the raw material.

### (1-4-4) Monomer Ratio Analysis of Trastuzumab-Affinity Peptide Conjugate

The trastuzumab-affinity peptide conjugate obtained in (1-4-1) was subjected to size exclusion chromatography (SEC) analysis according to Example 1-1-4.

As a result, although a conjugation reaction was performed in an FMR, the monomer ratio exceeded 99%.

### (1-5) Synthesis of Rituximab-Affinity Peptide Conjugate Using Affinity Peptide Reagent (1)

### (1-5-1) Conjugation of Affinity Peptide Reagent (1) and Rituximab

For anti-CD20 IgG antibody rituximab (Roche), a conjugation reaction was attempted using the affinity peptide reagent (1). Condition 2 in Table 3 in (1-1-1) was selected as the reaction condition.

### (1-5-2) DAR Analysis of Rituximab-Affinity Peptide Conjugate

ESI-TOFMS analysis of the rituximab-affinity peptide conjugate obtained in (1-5-1) was performed according to the condition in Example 1-1-2. For the raw material rituximab, a peak was observed at 147,404. For a reaction product, a peak was observed at 152,203 with two modifying reagents (1) introduced.

The determination of the peptide/antibody binding ratio using DAR calculator (software from Agilent Technologies, Inc.) was subsequently performed and the result is listed in Table 3. The average peptide/antibody binding ratio calculated from the DAR peak in Table 3 and % Area was 2.0.

### (1-5-3) Determination of Heavy Chain Selectivity of Rituximab-Affinity Peptide Conjugate

ESI-TOFMS analysis under reduction conditions of the rituximab-affinity peptide conjugate obtained in (1-5-1) was performed according to the condition in Example 1-1-3. For the raw material rituximab, heavy chain peaks were observed at 50,507 and 50,670 and a light chain peak was observed at 23,036, and for a reaction product, peaks were observed at 52,909 and 53,070 with a linker introduced to the heavy chain, and a light chain peak was observed at 23,036, the same as that of the raw material.

### (1-5-4) Monomer Ratio Analysis of Rituximab-Affinity Peptide Conjugate

The rituximab-affinity peptide conjugate obtained in (1-5-1) was subjected to size exclusion chromatography (SEC) analysis according to Example 1-1-4.

As a result, although a conjugation reaction was performed in an FMR, the monomer ratio exceeded 99%.

### Example 2: Integration of Regioselective Modification Reaction of Antibody by Affinity Peptide Reagent and Subsequent Reduction Reaction

(2-1) Integration of Regioselective Modification Reaction of Antibody using Affinity Peptide (3) and Subsequent Reduction Reaction

The first reservoir (1) of the FMR (FIG. 7) was filled with an anti-HER2 IgG antibody trastuzumab (Chugai Pharmaceutical Co., Ltd.) 10 mg/mL 50 mM sodium acetate buffer solution (pH=5.5). The second reservoir (2) was filled with a 0.667 mM solution of an affinity peptide 3 (compound 3) with a conjugation buffer solution (acetate:DMF=9:1, pH 5.5). The third reservoir (3) was filled with a 0.667 mM TCEP solution. The antibody solution introduced at a flow rate of 1.0 mL/min and the affinity peptide solution introduced at a flow rate of 1.0 mL/min were mixed in the first micromixer (M1) and subjected to a conjugation reaction in the first reaction tube (R1) for a reaction time of 0.166 minute. Subsequently, the reaction solution flowing at a flow rate of 2.0 mL/min in the first reaction tube (R1) and the TCEP solution introduced at a flow rate of 2.0 mL/min were mixed in the second micromixer (M2) and subjected to a reduction reaction in the second reaction tube (R2) for 1.5 minutes. The solution after the conjugation reaction in the second reaction tube (R2) was collected by the fraction collector.

### (2-2) Analysis of Reduced Antibody

ESI-TOFMS analysis of the reduced antibody obtained in (2-1) was performed based on the condition in Example 1-1-3, in which pretreatment by a reducing agent was omitted. For the raw material trastuzumab, heavy chain peaks were observed at 50,594 and 50,755 and a light chain peak was observed at 23,439, and for a reaction product, peaks were observed at 50,683 and 50,845 with a linker introduced to the heavy chain, and a light chain peak was observed at 23,439, the same as that of the raw material.

### Example 3: Integration of Regioselective Modification Reaction of Antibody Using Affinity Peptide (5) and ADC Synthesis

(3-1) Regioselective Modification Reaction of Antibody using Affinity Peptide (5) and ADC Synthesis

The first reservoir (1) of the FMR (FIG. 8) was filled with an anti-HER2 IgG antibody trastuzumab (Chugai Pharmaceutical Co., Ltd.) 10 mg/mL 50 mM sodium acetate buffer solution (pH=5.5). The second reservoir (2) was filled with a 0.333 mM solution of an affinity peptide 5 (compound 5) in the previous report (WO 2019/240288 (WO 2019/240288A1), Example 84-1-2) with a conjugation buffer solution (acetate:DMF=9:1, pH 5.5). The third reservoir (3) was filled with a 0.50 mM solution of a payload DBCO-MMAE (from Abzena, compound 6) with a conjugation buffer solution (acetate:DMA=9:1, pH 5.5). The antibody solution introduced at a flow rate of 1.0 mL/min and the affinity peptide solution introduced at a flow rate of 1.0 mL/min were mixed in the first micromixer (M1) and subjected to a conjugation reaction in the first reaction tube (R1) for a reaction time of 3 minutes. Subsequently, the reaction solution flowing at a flow rate of 2.0 mL/min in the first reaction tube (R1) and the payload solution introduced at a flow rate of 2.0 mL/min were mixed in the second micromixer (M2) and subjected to a conjugation reaction in the second reaction tube (R2) for 1.5 minutes to yield an ADC. The solution after the conjugation reaction in the second reaction tube (R2) was collected by the fraction collector.

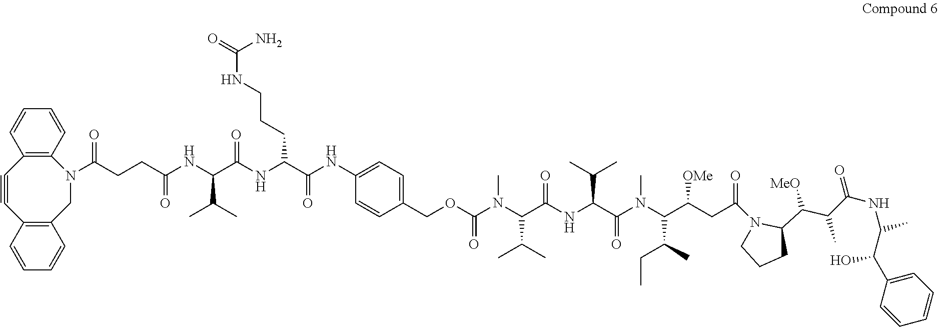

(Amino acid sequence of peptide portion: SEQ ID NO: 12)

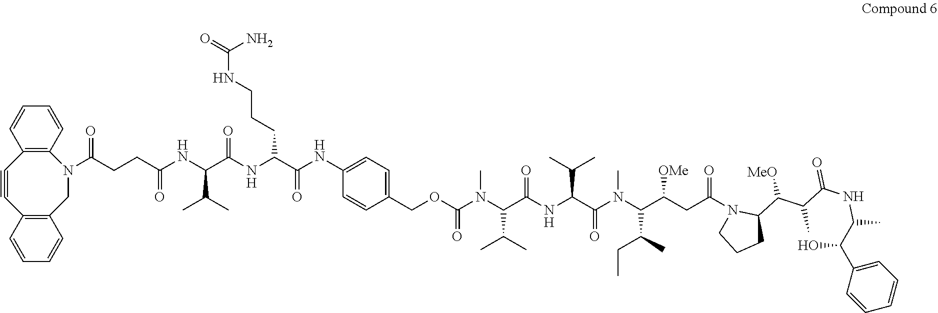

### (3-2) Analysis of ADC

ESI-TOFMS analysis of the ADC obtained in (3-1) was performed according to the previous report (WO 2019/240288 (WO 2019/240288A1), which are incorporated herein by reference in their entireties). The product was confirmed as a mass number of 151,336 with two MMAEs introduced to trastuzumab, in the same manner as the previous report (WO 2019/240288 (WO 2019/240288A1), which are incorporated herein by reference in their entireties, Example 12-2-1).

### (3-3) Determination of Heavy Chain Selectivity of ADC

ESI-TOFMS analysis under reduction conditions of the ADC obtained in (3-1) was performed according to the previous report (WO 2019/240288 (WO 2019/240288A1), which are incorporated herein by reference in their entireties). For the trastuzumab-MMAE conjugate, peaks were observed at 52,154 and 52,316 with MMAE introduced to the heavy chain and a light chain peak was observed at 23,440, the same as that of the raw material. This indicated that the azide group introduction by the affinity peptide using the FMR proceeded regioselectively to the antibody, and that a regioselective ADC was synthesized by subsequent conjugation with the payload.

### (3-4) Monomer Ratio Analysis of Regioselective ADC

The regioselective ADC compound obtained in (3-1) was subjected to size exclusion chromatography (SEC) analysis according to Example 1-1-4. Although a conjugation reaction was in an FMR, the monomer ratio exceeded 98%.

### Example 4: Integration of Regioselective Modification Reaction of Antibody Using Affinity Peptide (1) and ADC Synthesis Following Linker Cleavage Reaction

(4-1) ADC Synthesis by Integration of Regioselective Modification Reaction of Antibody using Affinity Peptide (1) and Linker Cleavage Reaction Payload Conjugation

The first reservoir (1) of the FMR (FIG. 3) was filled with an anti-HER2 IgG antibody trastuzumab (Chugai Pharmaceutical Co., Ltd.) 10 mg/mL 50 mM sodium acetate buffer solution (pH=5.5). The second reservoir (2) was filled with a 0.667 mM solution of an affinity peptide 1 (compound 1) in the previous report (WO 2019/240287 (WO 2019/240287A1), Example 84) with a conjugation buffer solution (acetate:DMF=9:1, pH 5.5). The third reservoir (3) was filled with a 50 mM hydroxylamine solution. The fourth reservoir (4) was filled with a 0.25 mM solution of a payload DBCO-MMAE (from Abzena, compound 6) with a conjugation buffer solution (acetate:DMA=9:1, pH 5.5). The antibody solution introduced at a flow rate of 1.0 mL/min and the affinity peptide solution introduced at a flow rate of 1.0 mL/min were mixed in the first micromixer (M1) and subjected to a conjugation reaction in the first reaction tube (R1) for a reaction time of 3 minutes. Subsequently, the reaction solution flowing at a flow rate of 2.0 mL/min in the first reaction tube (R1) and the hydroxylamine solution introduced at a flow rate of 2.0 mL/min were mixed in the second micromixer (M2) and subjected to a linker cleavage reaction in the second reaction tube (R2) for 1.5 minutes. Subsequently, the reaction solution flowing at a flow rate of 4.0 mL/min in the second reaction tube (R2) and the payload solution introduced at a flow rate of 4.0 mL/min were mixed in the third micromixer (M3) and subjected to a conjugation reaction in the third reaction tube (R3) for 1.5 minutes to yield an ADC. The solution after the conjugation reaction in the third reaction tube (R3) was collected by the fraction collector.

### (4-2) Analysis of ADC

ESI-TOFMS analysis of the ADC obtained in (4-1) was performed according to the previous report (WO 2019/240288 (WO 2019/240288A1), which are incorporated herein by reference in their entireties), and the product was confirmed as a mass number of 151,625 with two MMAEs introduced to trastuzumab, in the same manner as in Example (12-2-1).

### (4-3) Determination of Heavy Chain Selectivity of ADC

ESI-TOFMS analysis under reduction conditions of the ADC obtained in (4-1) was performed according to the previous report (WO 2019/240288 (WO 2019/240288A1), which are incorporated herein by reference in their entireties). For the trastuzumab-MMAE conjugate, peaks were observed at 53,206 and 53,368 with MMAE introduced to the heavy chain and a light chain peak was observed at 23,440, the same as that of the raw material. This indicated that the azide group introduction by the affinity peptide using the FMR proceeded regioselectively to the antibody, and that a regioselective ADC was synthesized by subsequent conjugation with the payload.

### (4-4) Monomer Ratio Analysis of Regioselective ADC

The regioselective ADC compound obtained in (4-1) was subjected to size exclusion chromatography (SEC) analysis according to Example 1-1-4. As a result, although a conjugation reaction was performed in an FMR, the monomer ratio exceeded 99%.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

### SEQUENCE LISTING

1. A method for producing an intermediate antibody regioselectively modified, the method comprising:
(1) mixing a solution comprising an antibody material and a solution comprising a reagent for regioselectively modifying an antibody in a micromixer to form a mixture comprising the antibody material and the reagent,
wherein the reagent comprises a compound comprising an affinity substance to an antibody and a reactive group to an antibody; and
(2) passing the mixture through a reaction channel to allow the antibody material and the reagent to react in the reaction channel to form a solution comprising an intermediate antibody regioselectively modified,
wherein the processes (1) and (2) are continuously performed in a flow microreactor.

2. The method according to claim 1, wherein the antibody material is IgG.

3. The method according to claim 1, wherein the affinity substance is a peptide having affinity to a heavy chain constant region of an antibody.

4. The method according to claim 1, wherein the reactive group is a reactive group to an amino group.

5. The method according to claim 1, wherein the compound comprises the affinity substance, the reactive group, and a cleavable site, and optionally further comprises a bioorthogonal functional group.

6. The method according to claim 5, wherein the compound comprises the affinity substance, the reactive group, the cleavable site, and the bioorthogonal functional group, wherein
(i) the cleavable site is present at an affinity substance-side position between the affinity substance and the reactive group, and
(ii) the bioorthogonal functional group is present at a reactive group-side position between the affinity substance and the reactive group.

7. The method according to claim 5, wherein the compound comprises the affinity substance, the reactive group, and a cleavable site capable of forming a bioorthogonal functional group by cleavage,
wherein the cleavable site capable of forming a bioorthogonal functional group by cleavage is present at a position between the affinity substance and the reactive group.

8. The method according to claim 1, wherein the compound comprises the affinity substance, the reactive group, a leaving group, and a bioorthogonal functional group, wherein
(i) the leaving group and the reactive group are coupled with each other and present at a position between the affinity substance and the bioorthogonal functional group,
(ii) the leaving group is present at an affinity substance-side position between the affinity substance and the bioorthogonal functional group, and
(iii) the reactive group is present at a bioorthogonal functional group-side position between the affinity substance and the bioorthogonal functional group.

9. The method according to claim 1, wherein the micromixer is a collision-type micromixer.

10. The method according to claim 9, wherein the collision-type micromixer is a T-shaped micromixer.

11. The method according to claim 1, wherein
the solution comprising an antibody material is passed through a first inlet channel,
the solution comprising a reagent for regioselectively modifying an antibody is passed through a second inlet channel,
the micromixer is provided at a junction of the first inlet channel and the second inlet channel, and
both of a representative diameter ratio of the micromixer to the first inlet channel (micromixer/first inlet channel) and a representative diameter ratio of the micromixer to the second inlet channel (micromixer/second inlet channel) are 0.95 or less.

12. The method according to claim 1, wherein a residence time of the mixture in the reaction channel is less than 3 minutes.

13. The method according to claim 1, wherein a modification ratio of the compound to the intermediate antibody (modification by the compound/antibody) is 1.5 to 2.5 per immunoglobulin unit comprising two light chains and two heavy chains.

14. The method according to claim 1, wherein a monomer ratio of the intermediate antibody analyzed by size exclusion chromatography is 98% or more.

15. A method for producing an antibody derivative regioselectively having a bioorthogonal functional group or bioorthogonal functional groups, the method comprising:
(1) mixing a solution comprising an antibody material and a solution comprising a reagent for regioselectively modifying an antibody in a first micromixer to form a first mixture comprising the antibody material and the reagent,
wherein the reagent comprises a compound comprising an affinity substance to an antibody, a reactive group to an antibody, and a cleavable site, and optionally further comprising a bioorthogonal functional group;
(2) passing the first mixture through a first reaction channel to allow the antibody material and the reagent to react in the first reaction channel to form a solution comprising an intermediate antibody regioselectively modified,
wherein the intermediate antibody regioselectively modified is an intermediate antibody regioselectively having a structural unit comprising the affinity substance and the cleavable site and optionally further comprising a bioorthogonal functional group;
(3) mixing the solution comprising the intermediate antibody and a solution comprising a cleavage agent in a second micromixer to form a second mixture comprising the intermediate antibody and the cleavage agent; and
(4) passing the second mixture through a second reaction channel to allow the intermediate antibody and the cleavage agent to react in the second reaction channel to form a solution comprising an antibody derivative regioselectively having a bioorthogonal functional group or bioorthogonal functional groups,
wherein the bioorthogonal functional group in the antibody derivative is (a) the bioorthogonal functional group in the compound or (b) a bioorthogonal functional group formed by cleavage of the cleavable site by the cleavage agent,
wherein the processes (1) to (4) are continuously performed in a flow microreactor.

16. The method according to claim 15, wherein a total time of a residence time of the first mixture in the first reaction channel and a residence time of the second mixture in the second reaction channel is within 4.5 minutes.

17. The method according to claim 15, wherein a modification ratio of the bioorthogonal functional group to the antibody derivative regioselectively having a bioorthogonal functional group or bioorthogonal functional groups (bioorthogonal functional group/antibody) is 1.5 to 2.5 per immunoglobulin unit comprising two light chains and two heavy chains.

18. The method according to claim 15, wherein a monomer ratio of the antibody derivative regioselectively having a bioorthogonal functional group or bioorthogonal functional groups analyzed by size exclusion chromatography is 98% or more.

19. A method for producing an antibody derivative regioselectively having a functional substance or functional substances, the method comprising:
(I) forming (a) a solution comprising an intermediate antibody regioselectively having a bioorthogonal functional group by the method comprising:
(1) mixing a solution comprising an antibody material and a solution comprising a reagent for regioselectively modifying an antibody in a micromixer to form a mixture comprising the antibody material and the reagent,
wherein the reagent comprises a compound comprising an affinity substance to an antibody and a reactive group to an antibody; and
(2) passing the mixture through a reaction channel to allow the antibody material and the reagent to react in the reaction channel to form a solution comprising an intermediate antibody regioselectively modified,
wherein the processes (1) and (2) are continuously performed in a flow microreactor wherein the compound comprises the affinity substance, the reactive group, a leaving group, and a bioorthogonal functional group, wherein
(i) the leaving group and the reactive group are coupled with each other and present at a position between the affinity substance and the bioorthogonal functional group,
(ii) the leaving group is present at an affinity substance-side position between the affinity substance and the bioorthogonal functional group, and
(iii) the reactive group is present at a bioorthogonal functional group-side position between the affinity substance and the bioorthogonal functional group;
or forming (b) a solution comprising an antibody derivative regioselectively having a bioorthogonal functional group or bioorthogonal functional groups by a method comprising:
(1) mixing a solution comprising an antibody material and a solution comprising a reagent for regioselectively modifying an antibody in a first micromixer to form a first mixture comprising the antibody material and the reagent,
wherein the reagent comprises a compound comprising an affinity substance to an antibody, a reactive group to an antibody, and a cleavable site, and optionally further comprising a bioorthogonal functional group;
(2) passing the first mixture through a first reaction channel to allow the antibody material and the reagent to react in the first reaction channel to form a solution comprising an intermediate antibody regioselectively modified,
wherein the intermediate antibody regioselectively modified is an intermediate antibody regioselectively having a structural unit comprising the affinity substance and the cleavable site and optionally further comprising a bioorthogonal functional group;
(3) mixing the solution comprising the intermediate antibody and a solution comprising a cleavage agent in a second micromixer to form a second mixture comprising the intermediate antibody and the cleavage agent; and
(4) passing the second mixture through a second reaction channel to allow the intermediate antibody and the cleavage agent to react in the second reaction channel to form a solution comprising an antibody derivative regioselectively having a bioorthogonal functional group or bioorthogonal functional groups,
wherein the bioorthogonal functional group in the antibody derivative is (a) the bioorthogonal functional group in the compound or (b) a bioorthogonal functional group formed by cleavage of the cleavable site by the cleavage agent,
wherein the processes (1) to (4) are continuously performed in a flow microreactor;
(II) mixing the solution formed in (I) and a solution comprising a functional substance in a micromixer to form (a) a mixture comprising the intermediate antibody and the functional substance or (b) a mixture comprising the antibody derivative and the functional substance; and
(III) passing the mixture formed in (II) through a reaction channel to allow (a) the intermediate antibody and the functional substance or (b) the antibody derivative and the functional substance to react in the reaction channel to form a solution comprising an antibody derivative regioselectively having a functional substance or functional substances,
wherein the processes (I) to (III) are continuously performed in a flow microreactor.

20. The method according to claim 19, wherein the functional substance is a pharmaceutical, a labelling substance, or a stabilizer.

21. The method according to claim 19, wherein a total residence time in all reaction channels is within 6 minutes.

22. The method according to claim 19, wherein a modification ratio of the functional substance to the antibody derivative regioselectively having a functional substance or functional substances (functional substance/antibody) is 1.5 to 2.5 per immunoglobulin unit comprising two light chains and two heavy chains.

23. The method according to claim 19, wherein a monomer ratio of the antibody derivative regioselectively having a functional substance or functional substances analyzed by size exclusion chromatography is 98% or more.
